(19) United States
(12) Patent Application Publication
(10) Pub. No.: US 2019/0150858 A1
(43) Pub. Date: May 23, 2019

(54) IMAGE DISPLAY DEVICE AND IMAGE DISPLAY METHOD

(71) Applicant: Daiwa House Industry Co., Ltd., Osaka (JP)

(72) Inventors: Masahiko Ishikawa, Osaka (JP); Takashi Orime, Osaka (JP); Yoshiho Negoro, Osaka (JP); Tsukasa Nakano, Osaka (JP); Yasuo Takahashi, Osaka (JP)

(73) Assignee: Daiwa House Industry Co., Ltd., Osaka (JP)

(21) Appl. No.: 16/066,488

(22) PCT Filed: Dec. 26, 2016
(86) PCT No.: PCT/JP2016/088629

§ 371 Date: Jun. 27, 2018

(30) Foreign Application Priority Data
Dec. 28, 2015 (JP) 2015-256875
Jun. 10, 2016 (JP) 2016-116538

(51) Int. Cl. A61B 5/00; G09G 5/377; G06T 7/00; G16H 15/00

(52) U.S. Cl. CPC A61B 5/7425; G16H 15/00; G06T 7/0012; G09G 5/377

(57) ABSTRACT

An image display device includes: a controller that: acquires biological information of a subject measured by a sensor at a preset time interval; displays an image shot by a shooting device on a display; specifies a position of a figure image in the shot image; and detects a specified subject in a predetermined state. While detecting the specified subject, the controller acquires the biological information of the specified subject at a second time interval shorter than a first time interval which is the time interval in normal times. At the time of displaying the shot image including the figure image of the specified subject on the display, the controller displays information corresponding to the biological information of the specified subject acquired at the second time interval in a region corresponding to the specified position of the figure image of the specified subject while making the information overlap the figure image.

### TECHNICAL FIELD

The present invention relates to an image display device and an image display method, and relates to an image display device and an image display method for confirming an image of a subject in a predetermined state, and biological information of the subject.

### BACKGROUND ART

At the time of displaying an image of a certain subject on a display, display of biological information of the subject together is already known. An example of the display includes the technology described in Patent Literature 1. In a remote patient monitoring system described in Patent Literature 1, biological information of a patient is measured by a vital sensor, an image of the patient is shot by a video camera, images corresponding to a measurement result of the vital sensor are fitted into and combined with the image of the patient, and the combined composite image is displayed. Thereby, it is possible to easily confirm behaviors and the biological information of the patient in a remote place.

### CITATION LIST

### Patent Literature

PATENT LITERATURE 1: JP 11-151210

In the system described in Patent Literature 1, a display position of the biological information in the composite image (measurement result of the vital sensor) is fixed. When the display position of the biological information is fixed in this way, and for example in a case where images of plural patients are presented, it is difficult to distinguish whose information the displayed biological information relates to.

For example, in a case where biological information of a patient is radically changed due to a sudden change in a condition of the patient, exercise of the patient, etc., there is a need for catching the change as soon as possible. Meanwhile, as a time interval (acquisition span) at which the biological information is acquired is longer, it is more difficult to realize the change in the biological information.

Further, when images and biological information of plural persons are displayed, there is a need for associating the biological information of each person with the image (figure image) of each person to display. That is, in a case where images and biological information of plural persons are displayed, association relationships between the biological information and figure images, specifically, whose information the acquired biological information relates to, and where the figure image of the person is displayed are specified. As a matter of course, there is a need for properly specifying association relationships between the biological information and the figure images.

### SUMMARY

An image display device and an image display method according to one or more embodiments of the present invention are capable of, at the time of confirming biological information of a subject in a predetermined state together with an image of the subject, more properly confirming the biological information.

Upon displaying figure images and biological information of plural subjects, the image display device and the image display method according to one or more embodiments of the present invention properly specify association relationships between the biological information and the figure images.

An image display device according to one or more embodiments of the present invention comprises (A) a biological information acquiring section that acquires biological information of a subject measured by a sensor at a preset time interval, (B) an image displaying section that displays an image being shot by a shooting device on a display, (C) a position specifying section that specifies a position of a figure image included in the image, the position within the image, and (D) a detecting section that detects a specified subject who is the subject in a predetermined state. (E) While the detecting section is detecting the specified subject, the biological information acquiring section acquires the biological information of the specified subject at a second time interval shorter than a first time interval which is the time interval in normal times, and (F) at the time of displaying the image including the figure image of the specified subject on the display, the image displaying section displays information corresponding to the biological information of the specified subject acquired at the second time interval by the biological information acquiring section in a region corresponding to the position of the figure image of the specified subject, the position being specified by the position specifying section while overlapping with the image.

In the image display device according to one or more embodiments of the present invention formed as above, at the time of displaying the image including the figure image of the specified subject on the display, the information corresponding to the biological information of the specified subject is displayed in the region in the image corresponding to the specified position of the figure image of the specified subject while overlapping with the image. Thereby, whose information the biological information displayed on the display relates to is easily grasped. In the image display device according to one or more embodiments of the present invention, while the detecting section is detecting the specified subject, the biological information of the specified subject is acquired at the time interval shorter than the time interval in normal times. Thereby, at the time of a change in the biological information of the specified subject, it is possible to more promptly catch the change.

In the image display device according to one or more embodiments of the present invention, the image displaying section updates contents of information displayed as the information corresponding to the biological information of the specified subject while overlapping with the image every time the biological information acquiring section acquires the biological information of the specified subject.

With the above configuration, the information corresponding to the latest biological information is displayed on the display. Thus, at the time of the change in the biological information of the specified subject, it is possible to further promptly catch the change.

In the image display device according to one or more embodiments of the present invention, the sensor measures the biological information whose magnitude is changed according to an activity degree of the subject wearing the sensor, and is capable of communicating with the image display device, the image display device has an identifying section that identifies the specified subject detected by the detecting section, and a storing section that stores identification information of the sensor worn by the subject by each subject, and the biological information acquiring section reads the identification information of the sensor associated with the specified subject who is identified by the identifying section out of the storing section, and by communicating with the sensor specified by the read identification information, acquires the biological information of the specified subject.

With the above configuration, when a specified subject is detected, the specified subject is identified. After that, by communicating with the sensor worn by the identified specified subject, the biological information of the specified subject is acquired. With such procedure, it is possible to more reliably acquire the biological information of the detected specified subject.

In the image display device according to one or more embodiments of the present invention, the detecting section detects the specified subject in a predetermined place, and the image displaying section displays the image on the display installed in a place separated from the predetermined place.

With the above configuration, it is possible to confirm an image and biological information of a subject staying in a certain place in a remote place separated from the place.

The image display device according to one or more embodiments of the present invention comprises a change detecting section that detects a change in at least one of a face position, a facial direction, and a line of sight of an image confirming person who is in front of the display and confirms the figure image of the specified subject on the display, wherein when the change detecting section detects the change, a range of the image shot by the shooting device, the range being displayed on the display by the image displaying section is shifted according to the change.

With the above configuration, the range of the image shot by the shooting device, the range being displayed on the display is shifted in conjunction with the change in the face position, the facial direction, and the line of sight of the image confirming person. Thereby, for example, in a case where a lot of specified subjects are detected and even when the number of specified subjects to be displayed on the display at once is limited, it is possible to confirm the image and the biological information of all the specified subjects by the image confirming person changing the face position, the direction, or the line of sight.

In the image display device according to one or more embodiments of the present invention, at the time of displaying the information corresponding to the biological information of the specified subject while overlapping with the image, the image displaying section determines whether or not the biological information of the specified subject satisfies preset conditions, and displays the information corresponding to the biological information of the specified subject in a display mode corresponding to a determination result.

With the above configuration, it is determined whether or not the biological information of the specified subject satisfies the preset conditions, and the information corresponding to the biological information of the specified subject is displayed in the display mode corresponding to the determination result. Thereby, when the biological information of the specified subject satisfies the predetermined conditions (for example, when the biological information is of contents in abnormal times), it is possible to easily remind of such a situation.

The image display device according to one or more embodiments of the present invention further comprises an information analyzing section that adds up the biological information acquired at the first time interval in normal times by the biological information acquiring section for each subject and analyzes the biological information for each subject, wherein the image displaying section determines whether or not the biological information of the specified subject satisfies a condition associated with the specified subject among the conditions set for each subject according to an analysis result of the information analyzing section.

With the above configuration, the condition by which determination is made for the biological information of the specified subject is set for each subject based on biological information acquired in normal times. By setting the condition in this way, at the time of determining whether or not the biological information of the specified subject satisfies the condition, it is possible to additionally involve the biological information of the specified subject in normal times. That is, for example, upon determining whether or not the biological information of the specified subject is of the contents in abnormal times, it is possible to consider an individual difference of the specified subject.

In the image display device according to one or more embodiments of the present invention, when the detecting section detects plural specified subjects, the biological information acquiring section acquires the biological information of the specified subjects measured by the sensors and other information relating to the specified subjects respectively from transmitters prepared for each specified subject, the transmitters on which the sensors are mounted, the position specifying section executes first processing of specifying the position of the figure image of the specified subject who has performed a predetermined action, and second processing of specifying the transmitter which sent the other information relating the specified subject who has performed the predetermined action among the transmitters for each specified subject, and at the time of displaying the image including the figure image of the specified subject who has performed the predetermined action on the display, the image displaying section displays information corresponding to the biological information acquired by the biological information acquiring section from the transmitter specified by the position specifying section in the second processing in a region corresponding to the position specified by the position specifying section in the first processing while overlapping with the image.

With the above configuration, regarding the specified subject who has performed the predetermined action among the plural specified subjects, where in the image displayed on the display the figure image of the above specified subject is displayed (that is, the display position) is specified. The transmitter which sent the information (other information) relating the specified subject who has performed the predetermined action among the transmitters prepared for each specified subject is specified. Thereby, it is possible to specify an association relationship between the figure image of the specified subject who has performed the predetermined action and the biological information of the specified subject. As a result of specifying of the association relationship between the figure image and the biological information in this way, the biological information of the specified subject is displayed in the region corresponding to the position of the figure image of the specified subject who has performed the predetermined action. As above, even in a case where there are plural specified subjects, it is possible to associate the biological information of each specified subject with the figure image of the specified subject to display.

In the image display device according to one or more embodiments of the present invention, the biological information acquiring section acquires, respectively from the transmitters prepared for each specified subject, action information generated at the time of detecting actions of the specified subjects by action detectors mounted on the transmitters as the other information, and in the second processing, the position specifying section specifies the transmitter which sent the action information generated at the time of detecting the predetermined action by the action detector among the transmitters for each specified subject.

With the above configuration, the action information generated at the time of detecting the actions of the specified subjects by the action detectors mounted on the transmitters is acquired as the other information relating to the specified subjects. With such a configuration, when a certain specified subject performs a predetermined action, action information generated at the time of detecting the predetermined action by an action detector is sent from a transmitter prepared for the specified subject. Thereby, it is possible to more precisely specify the transmitter of the specified subject who has performed the predetermined action. As a result, it is possible to more properly specify the association relationship between the figure image and the biological information of each specified subject.

The image display device according to one or more embodiments of the present invention comprises a control information sending section that sends control information for controlling a device installed in a place where there are the plural specified subjects, wherein the control information sending section sends the control information for controlling the device so that one of the plural specified subjects is encouraged to perform the predetermined action.

With the above configuration, it is possible to encourage one of the plural specified subjects to perform the predetermined action. Thereby, the position of the figure image of the specified subject who is performing the predetermined action, and the transmitter of the specified subject who is performing the predetermined action are more easily specified.

In the image display device according to one or more embodiments of the present invention, the biological information acquiring section acquires names of the specified subjects as the other information respectively from the transmitters prepared for each specified subject, the control information sending section sends the control information for making the device generate a sound indicating the name of the one of the plural specified subjects as the control information for controlling the device so that the one of the plural specified subjects is encouraged to perform the predetermined action, and in the first processing, the position specifying section specifies the position of the figure image of the specified subject who has performed a response action to the sound.

With the above configuration, in order to encourage the one of the plural specified subjects to perform the predetermined action, the sound indicating the name of the specified subject is generated. When the response action to this sound serves as the predetermined action, the position of the figure image of the specified subject who is performing the predetermined action, and the transmitter of the specified subject who is performing the predetermined action are markedly easily specified.

In the image display device according to one or more embodiments of the present invention, in the first processing, the position specifying section specifies the position of the figure image of the specified subject who is performing the predetermined action based on data indicating distances between body parts of the specified subject whose figure image is presented in the image and a reference position set in a place where the specified subject stays.

With the above configuration, the position specifying section specifies the position of the figure image of the specified subject who is performing the predetermined action based on the data indicating depth of the body parts of the specified subject whose figure image is presented in the image displayed on the display (depth will be described later). Thereby, it is possible to precisely specify the position of the figure image of the specified subject who is performing the predetermined action.

An image display method according to one or more embodiments of the present invention comprises the steps of (A) a computer acquiring biological information of a subject measured by a sensor at a preset time interval, (B) the computer displaying an image being shot by a shooting device on a display, (C) the computer specifying a position of the figure image included in the image, the position within the image, and (D) the computer detecting a specified subject who is the subject in a predetermined state. (E) While detecting the specified subject, the biological information of the specified subject is acquired at a second time interval shorter than a first time interval which is the time interval in normal times, and (F) at the time of displaying the image including the figure image of the specified subject on the display, information corresponding to the biological information of the specified subject acquired at the second time interval is displayed in a region corresponding to the specified position of the figure image of the specified subject while overlapping with the image.

With the above method, at the time of confirming the image of the subject in the predetermined state together with the biological information of the subject, it is possible to easily grasp whose biological information the biological information is. At the time of a change in the biological information, it is possible to more promptly catch the change.

According to one or more embodiments of the present invention, at the time of confirming the image of the subject in the predetermined state together with the biological information of the subject, it is possible to easily grasp whose biological information the biological information is. Thereby, for example, in a situation where the image of the plural specified subjects is displayed and even when each specified subject moves, the display position of the biological information is also changed according to the position after moving. Thus, it is possible to easily confirm the biological information of each subject after moving.

Additionally, according to one or more embodiments of the present invention, at the time of the change in the biological information due to a sudden change in a condition of the specified subject, exercise of the specified subject, etc., it is possible to more promptly catch the change. As a result, it is possible to promptly address the change in the biological information, and to maintain a favorable state of the specified subject (such as a health condition).

Additionally, according to one or more embodiments of the present invention, upon displaying the figure image and the biological information of each of the plural subjects, by associating the biological information of the specified subject who is performing the predetermined action with the figure image, it is possible to properly specify the association relationship between the biological information and the figure image.

### BRIEF DESCRIPTION OF DRAWINGS

FIG. 1 is an illustrative view of a communication system including an image display device according to one or more embodiments of the present invention.

FIG. 2 is a view showing a device configuration of a communication system including the image display device according to one or more embodiments of the present invention.

FIG. 3 is a view showing an image display unit installed on the subject side according to one or more embodiments of the present invention.

FIG. 4 is an illustrative view of depth data according to one or more embodiments of the present invention.

FIG. 5 is a view showing an image display unit installed on the image confirming person side according to one or more embodiments of the present invention.

FIG. 6 is a view showing a state where a displayed image according to an action of the image confirming person according to one or more embodiments of the present invention.

FIG. 7 is a view showing a functional configuration of the image display device according to one or more embodiments of the present invention.

FIG. 8 is a view showing a sensor ID storage table according to one or more embodiments of the present invention.

FIG. 9 is a view showing an average number of heartbeat storage table according to one or more embodiments of the present invention.

FIG. 10 is a view showing a flow of an exercise instruction flow according to one or more embodiments of the present invention.

FIG. 11 is a view showing a flow of image display processing (No. 1) according to one or more embodiments of the present invention.

FIG. 12 is a view showing the flow of the image display processing (No. 2) according to one or more embodiments of the present invention.

FIG. 13 is a view showing a device configuration of a communication system including an image display device according to one or more embodiments of the present invention.

FIG. 14 is a view showing a configuration of the image display device according to one or more embodiments of the present invention.

FIG. 15 is a view showing exchanges of various information according to one or more embodiments of the present invention.

FIG. 16 is a view showing a state where one of plural specified subjects is performing a predetermined action according to one or more embodiments of the present invention.

FIG. 17 is a view showing procedure of specifying a position of a figure image of the specified subject who is performing the predetermined action according to one or more embodiments of the present invention.

FIG. 18 is a view showing a flow of an association process according to one or more embodiments of the present invention.

### DETAILED DESCRIPTION OF EMBODIMENTS

Hereinafter, embodiments of the present invention will be described. The embodiments to be described below are examples not limiting the present invention but facilitating understanding of the present invention. That is, the present invention can be modified and improved without departing from the gist thereof, and equivalents thereof are included in the present invention, as a matter of course.

### <<Use of Image Display Device>>

An image display device according to one or more embodiments of the present invention is used by an image confirming person for confirming an image of a subject staying in a remote place. In particular, in one or more embodiments of the present invention, the image display device is utilized for building up a communication system for exercise instruction (hereinafter, the exercise instruction system 1).

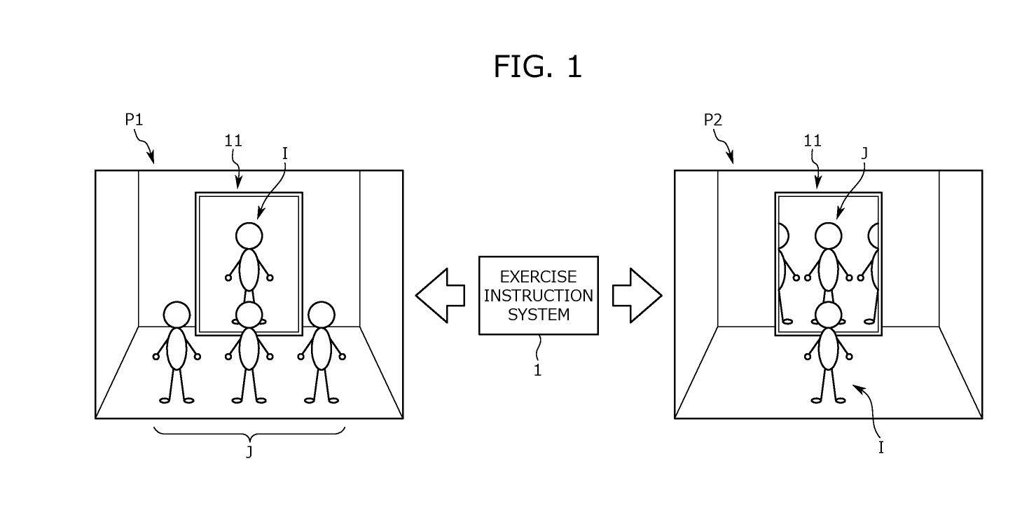

The above exercise instruction system 1 will be described with reference to FIG. 1. The exercise instruction system 1 is utilized by an instructor I serving as the image confirming person and participants J serving as the subject. With the exercise instruction system 1, the instructor I and the participants J can confirm images of one another in real time while staying in places (rooms) different from each other.

Specifically speaking, as shown in FIG. 1, the participants J can receive instructions (lesson) of the instructor I while watching the image of the instructor I on a display 11 installed in a gym P1. Meanwhile, as shown in the same figure, the instructor I can confirm the image of the participants J participating in the lesson on a display 11 installed in a dedicated booth P2 and monitor a state of the participants J (for example, a degree of understanding of the instructions, a fatigue degree, adequacy of physical movement, etc.)

The gym P1 corresponds to the “predetermined place” in one or more embodiments of the present invention, and the dedicated booth P2 corresponds to the “place separated from the predetermined place”. The above two places may be places respectively set in different buildings from each other, or may be set in rooms separated from each other in the same building.

By functions of the exercise instruction system 1, the instructor I can confirm current biological information together with a real-time image of the participants J participating in the lesson. The “biological information” indicates a characteristic amount to be changed according to the state of the participants J (health condition) or a physical condition, and in one or more embodiments of the present invention, indicates information whose magnitude is changed according to an activity degree (strictly, an exercise amount), specifically, the number of heartbeat. However, the present invention is not limited to this. For example, a breathing amount, consumed calories, or a body temperature change amount may be confirmed as the biological information.

In one or more embodiments of the present invention, wearable sensors 20 are used as sensors that measure the biological information of the participants J. The participants J can wear the wearable sensors 20, and an appearance of the wearable sensors is, for example, a wristband shape. The participants J wear the wearable sensors 20 on a daily basis. Therefore, the biological information of the participants J (specifically, the number of heartbeat) is measured not only during the lesson but also in other times. Measurement results of the wearable sensors 20 are sent toward a predetermined destination via a communication network. The measurement results of the wearable sensors 20 may be directly sent from the wearable sensors 20 or may be sent via communication devices held by the participants J such as smartphones or cellular phones.

### <<Device Configuration of Exercise Instruction System>>

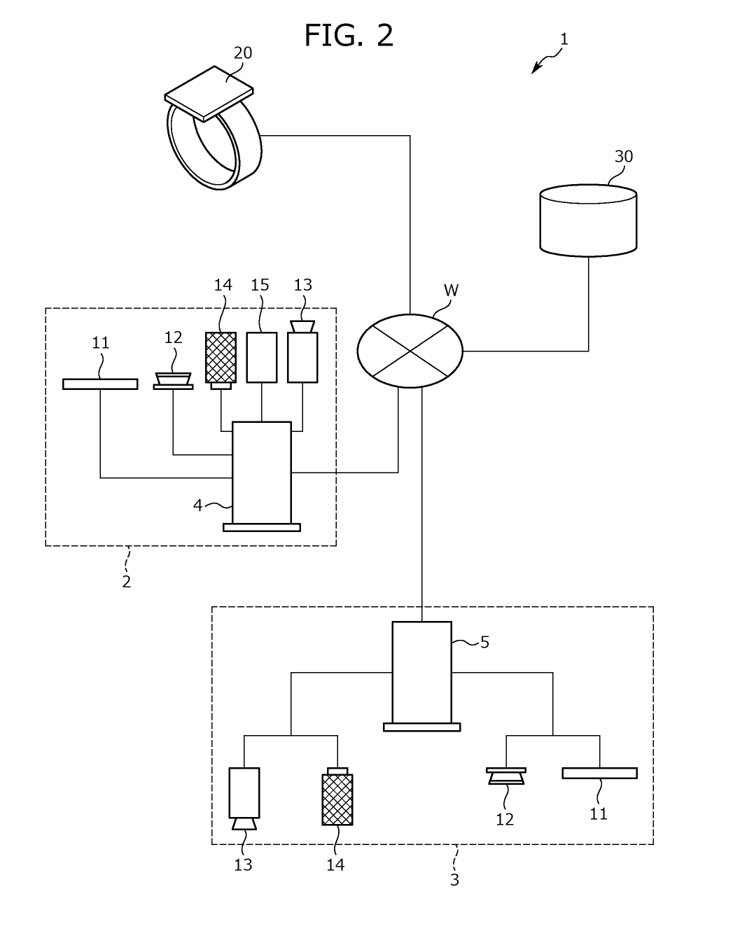

Next, a device configuration of the exercise instruction system 1 will be described with reference to FIG. 2. As shown in FIG. 2, the exercise instruction system 1 is formed by plural devices connected to the communication network (hereinafter, referred to as the network W). Specifically speaking, an image display unit utilized mainly by the participants J (hereinafter, referred to as the participant side unit 2), and an image display unit utilized mainly by the instructor I (hereinafter, referred to as the instructor side unit 3) are major constituent devices of the exercise instruction system 1.

As shown in FIG. 2, constituent devices of the exercise instruction system 1 include the wearable sensors 20 described above, and a biological information storage server 30. Each of the wearable sensors 20 is prepared for each participant J. In other words, each participant J wears the dedicated wearable sensor 20. The wearable sensor 20 regularly measures the number of heartbeat of the participant J wearing this sensor, and outputs the measurement result thereof. The biological information storage server 30 is a database server that receives the measurement results of the wearable sensors 20 via the network W, and stores the received measurement results by each participant J.

The wearable sensors 20 and the biological information storage server 30 are respectively connected to the network W, and are capable of communicating with devices connected to the network W (for example, a second data processing terminal 5 to be described later).

Hereinafter, detailed configurations of the participant side unit 2 and the instructor side unit 3 will be respectively described. First, the configuration of the participant side unit 2 will be described. The participant side unit 2 is used in the gym P1, and displays the image of the instructor I on the display 11 installed in the gym P1 and shoots the image of the participants J staying in the gym P1.

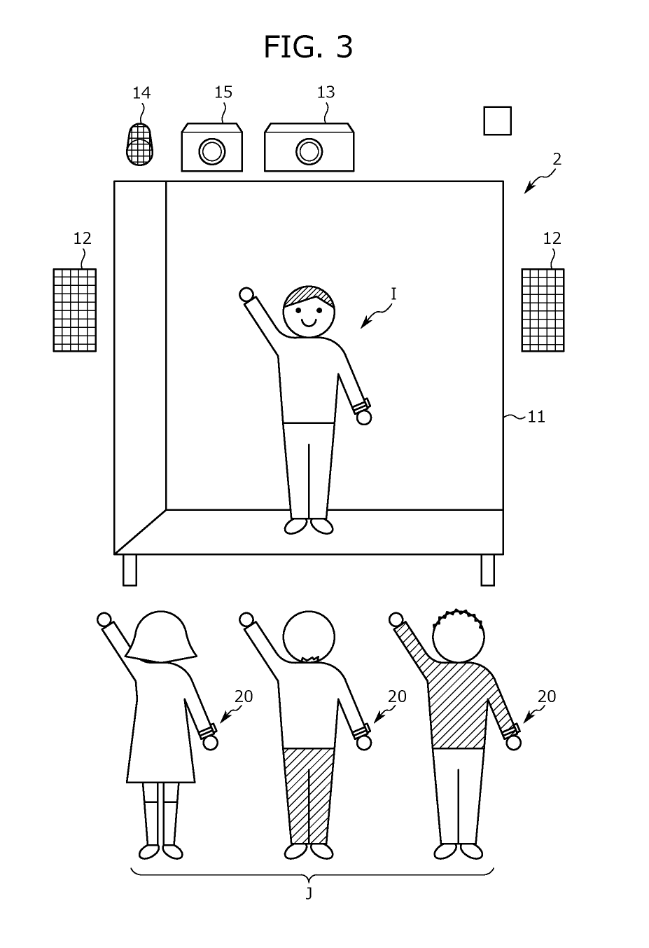

As shown in FIG. 2, the participant side unit 2 has a first data processing terminal 4, the display 11, a speaker 12, a camera 13, a microphone 14, and an infrared sensor 15 as constituent elements. The display 11 forms a screen for displaying the image. As shown in FIG. 3, the display 11 according to one or more embodiments of the present invention has screen size which is sufficient for displaying a figure image of the instructor I by life-size.

The speaker 12 is a device that generates a reproduced sound of the time when a sound embedded in the image is reproduced, the device being formed by a known speaker.

The camera 13 is an imaging device that shoots an image of an object within an imaging range (field angle), the imaging device being formed by a known network camera. The “image” indicates a collection of plural continuous frame images, that is, a video. In t one or more embodiments of the present invention, as shown in FIG. 3, the camera 13 provided in the participant side unit 2 is installed at a position immediately above the display 11. Therefore, the camera 13 provided in the participant side unit 2 shoots an image of an object at a position in front of the display 11 in operation.

Regarding the camera 13 provided in the participant side unit 2, the field angle is set to be relatively wide. That is, the camera 13 provided in the participant side unit 2 can shoot within a laterally (horizontally) wide range, and in a case where there are plural (for example, three or four) participants J in front of the display 11, can shoot the plural participants J at the same time.

The microphone 14 is to collect a sound in the room where the microphone 14 is installed.

The infrared sensor 15 is a so-called depth sensor, the sensor for measuring depth of a measurement object by the infrared method. Specifically speaking, the infrared sensor 15 emits an infrared ray toward the measurement object, and by receiving a reflected light thereof, measures depth of parts of the measurement object. The “depth” indicates a distance from a reference position to the measurement object (that is, a depth distance). In one or more embodiments of the present invention, a preset position in the gym P1 where the participants J stay, more specifically, a position of an image display surface (screen) of the display 11 installed in the gym P1 corresponds to the reference position. That is, the infrared sensor 15 measures, as depth, a distance between the screen of the display 11 and the measurement object, more strictly, a distance in the normal direction of the screen of the display 11 (in other words, the direction passing through the display 11).

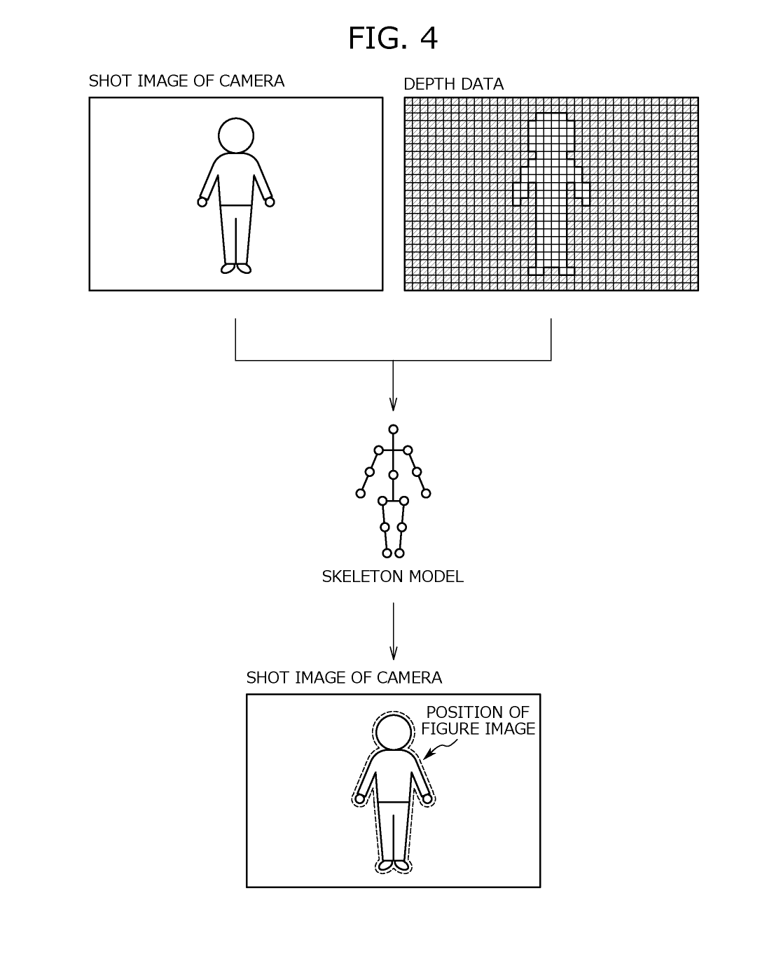

The infrared sensor 15 according to one or more embodiments of the present invention measures depth for each pixel of the time when the image shot by the camera 13 is divided into the predetermined number of pixels. By compiling measurement results of depth obtained for each pixel by an image, depth data for the image can be obtained. The depth data will be described with reference to FIG. 4. The depth data regulates depth by each pixel for the image shot by the camera 13 (strictly, each frame image). Specifically speaking, pixels hatched in the figure correspond to pixels belonging to the background image, and white pixels correspond to pixels belonging to the image of the object (for example, the figure image) placed on the front side of the background. Therefore, the depth data of the image including the figure image serves as data indicating depth of body parts of the person whose figure image is presented (distance from the reference position).

The first data processing terminal 4 is a device centering the participant side unit 2, the device being formed by a computer. A configuration of this first data processing terminal 4 is known and the first data processing terminal is formed by a CPU, memories such as a ROM and a RAM, a communication interface, a hard disk drive, etc. A computer program for executing a series of processing regarding image display (hereinafter, referred to as the first program) is installed in the first data processing terminal 4.

By starting up the first program, the first data processing terminal 4 controls the camera 13 and the microphone 14 to shoot the image in the gym P1 and collect the sound. The first data processing terminal 4 embeds the sound collected by the microphone 14 into the image shot by the camera 13, and then sends the image toward the instructor side unit 3. At this time, the first data processing terminal 4 also sends the depth data obtained by depth measurement of the infrared sensor 15.

By starting up the first program, the first data processing terminal 4 controls the display 11 and the speaker 12 at the time of receiving the image sent from the instructor side unit 3. Thereby, on the display 11 in the gym P1, an image of the interior of the dedicated booth P2 including the figure image of the instructor I is displayed. From the speaker 12 in the gym P1, a reproduced sound of the sound collected in the dedicated booth P2 (specifically, the sound of the instructor I) is emitted.

Next, the configuration of the instructor side unit 3 will be described. The instructor side unit 3 is used in the dedicated booth P2, and displays the image of the participants J participating in the lesson on the display 11 installed in the dedicated booth P2 and shoots the image of the instructor I staying in the dedicated booth P2.

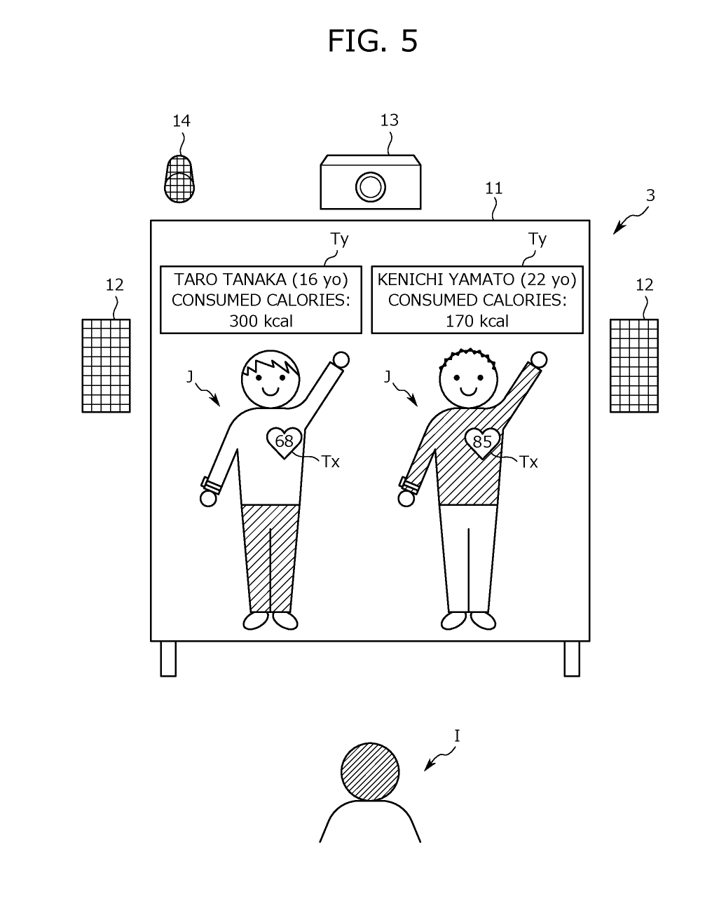

As shown in FIG. 2, the instructor side unit 3 has the second data processing terminal 5, the display 11, a speaker 12, a camera 13, and a microphone 14 as constituent elements. Configurations of the display 11, the speaker 12, the camera 13, and the microphone 14 are the substantially same as those provided in the participant side unit 2. As shown in FIG. 5, the display 11 according to one or more embodiments of the present invention has screen size which is sufficient for displaying figure images of the participants J by life-size. In one or more embodiments of the present invention, the display 11 provided in the instructor side unit 3 forms a slightly horizontally long screen, and as shown in FIG. 5, for example, can display the whole bodies of two participants J standing side by side at the same time.

The second data processing terminal 5 is a device centering the instructor side unit 3, the device being formed by a computer. This second data processing terminal 5 functions as the image display device according to one or more embodiments of the present invention. A configuration of the second data processing terminal 5 is known and the second data processing terminal is formed by a CPU, memories such as a ROM and a RAM, a communication interface, a hard disk drive, etc. A computer program for executing a series of processing regarding image display (hereinafter, referred to as the second program) is installed in the second data processing terminal 5.

By starting up the second program, the second data processing terminal 5 controls the camera 13 and the microphone 14 to shoot the image in the dedicated booth P2 and collect a sound. The second data processing terminal 5 embeds the sound collected by the microphone 14 into the image shot by the camera 13, and then sends the image toward the participant side unit 2.

By starting up the second program, the second data processing terminal 5 controls the display 11 and the speaker 12 at the time of receiving the image sent from the participant side unit 2. Thereby, on the display 11 in the dedicated booth P2, the image of the interior of the gym P1 including the figure images of the participants J is displayed. From the speaker 12 in the dedicated booth P2, a reproduced sound of the sound collected in the gym P1 (specifically, the sound of the participants J) is emitted.

The second data processing terminal 5 receives depth data together with the image from the participant side unit 2. By analyzing this depth data, in a case where the figure image is included in the image received from the participant side unit 2, the second data processing terminal 5 can specify a position of the figure image in the received image.

More specifically speaking, when the image including the figure image of the participant J participating in the lesson is sent from the participant side unit 2, the second data processing terminal 5 analyzes the depth data received together with the image. In such an analysis, the second data processing terminal 5 divides pixels constituting the depth data into pixels of the background image and pixels of the other image based on differences of depth. After that, the second data processing terminal 5 extracts pixels of the figure image from the pixels of the image other than the background image by applying a skeleton model of a person shown in FIG. 4. The skeleton model is a model simply showing a positional relationship regarding a head portion, a shoulder, elbows, wrists, the upper body center, a waist, knees, and ankles among a body of the person. A known method can be utilized as a method of obtaining the skeleton model.

Based on the pixels of the figure image extracted from the depth data, an image associated with the pixels is specified in the received image (image shot by the camera 13 of the participant side unit 2), and that image serves as the figure image. As a result, the position of the figure image in the received image is specified.

The method of specifying the position of the figure image is not limited to the method of specifying by using the depth data. For example, the position of the figure image may be specified by performing an image analysis on the image shot by the camera 13.

Based on the figure image specified from the received image, the depth data, and the skeleton model, the second data processing terminal 5 can detect a posture change and performance/non-performance of an action of a person whose figure image is presented (specifically, the participant J).

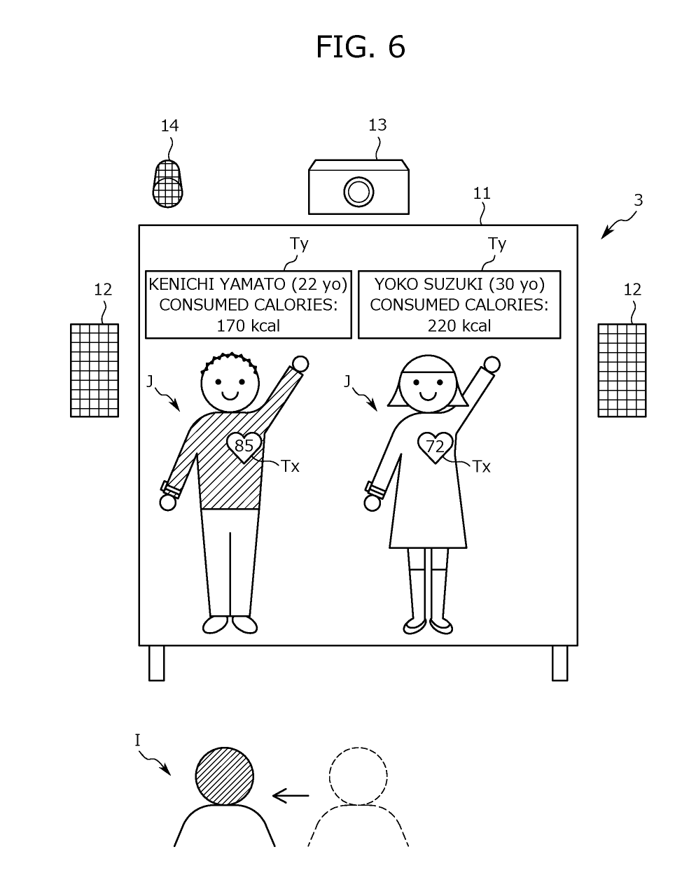

Further, while the camera 13 provided in the instructor side unit 3 is shooting the image of the instructor I, the second data processing terminal 5 detects a change in a face position of the instructor I by analyzing the shot image. When detecting the change in the face position of the instructor I, the second data processing terminal 5 shifts the image to be displayed on the display 11 installed in the dedicated booth P2 according to the face position after the change. Such image shifting will be described below with reference to FIGS. 5 and 6.

The image received by the second data processing terminal 5 from the participant side unit 2 is the image shot by the camera 13 which is installed in the gym P1. The image shot by the camera 13 which is installed in the gym P1 is, as described above, the image shot within the laterally (horizontally) wide range and the size thereof is slightly wider than the screen of the display 11 installed in the dedicated booth P2. Therefore, on the display 11 installed in the dedicated booth P2, part of the image received from the participant side unit 2 is displayed.

Meanwhile, when the face position of the instructor I is moved in the width direction of the display 11 (that is, in the lateral direction), the second data processing terminal 5 calculates a moving amount and the moving direction of the face position. After that, according to the calculated moving amount and moving direction, the second data processing terminal 5 shifts the image to be displayed on the display 11 installed in the dedicated booth P2 from the image displayed before movement of the face position of the instructor I. A specific example will be described. Supposing that when the image shown in FIG. 5 is displayed on the display 11 installed in the dedicated booth P2, the face position of the instructor I is moved rightward by a distance L. In such a case, the second data processing terminal 5 displays an image made by displacing the image shown in FIG. 5 leftward by an amount corresponding to the distance L, that is, the image shown in FIG. 6 on the display 11.

As described above, in one or more embodiments of the present invention, when the face of the instructor I who is confirming the image displayed on the display 11 in the dedicated booth P2 is laterally moved, the image displayed on the above display 11 is accordingly laterally displaced. That is, by the face of the instructor I being laterally moved, a range of the image received from the participant side unit 2, the range being displayed on the display 11 is shifted. As a result, by moving the face when watching a certain range of the image (for example, the image shown in FIG. 5) out of the image received from the participant side unit 2, the instructor I can watch, so-called look in a range of the image not displayed on the display 11 at that time point (for example, the image shown in FIG. 6). Thereby, in a case where there are a lot of participants J participating in the lesson in the gym P1 and even when the number of the participants to be displayed on the display 11 at once is limited, it is possible to confirm the image of all the participants J participating in the lesson by the instructor I changing the face position.

Further, the second data processing terminal 5 acquires the biological information of each participant J, that is, the number of heartbeat measured by the wearable sensor 20 worn by each participant J. A method of acquiring the number of heartbeat of the participant J will be described. An acquiring method in normal times is different from a method of the time of acquiring the number of heartbeat of the participant J participating in the lesson in the gym P1.

Specifically speaking, in normal times, the second data processing terminal 5 acquires the number of heartbeat of each participant J stored in the biological information storage server 30 by regularly communicating with the biological information storage server 30. It is possible to arbitrarily set a time interval at which the biological information is acquired from the biological information storage server 30 (hereinafter, referred to as the first time interval t1) but in one or more embodiments of the present invention, the time interval is set within a range of three to ten minutes.

Meanwhile, in a case where the number of heartbeat of the participant J participating in the lesson is acquired, the second data processing terminal 5 acquires the number of heartbeat of the participant J by directly communicating with the wearable sensor 20 worn by the participant J participating in the lesson. A time interval at which the biological information is acquired from the wearable sensor 20 (hereinafter, referred to as the second time interval t2) is set as a time shorter than the first time interval t1, and in one or more embodiments of the present invention set within a range of one to five seconds. This reflects the fact that the number of heartbeat of the participant J participating in the lesson is remarkably changed in comparison to normal times (when the participant does not participate in the lesson).

Further, at the time of displaying the figure image of the participant J participating in the lesson on the display 11, the second data processing terminal 5 displays information corresponding to the number of heartbeat in a region corresponding to the position of the figure image while overlapping with the figure image. More specifically speaking, as shown in FIGS. 5 and 6, a heart-shaped text box Tx in which the numerical value of the number of heartbeat (strictly, the numerical value indicating the measurement result of the wearable sensor 20) is described is displayed as pop-up in a region where a chest portion of the participant J participating in the lesson is presented.

The numerical value of the number of heartbeat described in text box Tx is updated every time the second data processing terminal 5 newly acquires the number of heartbeat. That is, the numerical value of the number of heartbeat described in text box Tx is updated at the time interval at which the number of heartbeat of the participant J participating in the lesson is acquired by the second data processing terminal 5 from the wearable sensor 20, that is, at the second time interval t2. As a result, the instructor I who is confirming the image of the participant J participating in the lesson on the display 11 can also confirm the current number of heartbeat of the participant J participating in the lesson.

In one or more embodiments of the present invention, the numerical value indicating the measurement result of the wearable sensor 20 is displayed as the information corresponding to the number of heartbeat. However, the present invention is not limited to this but similar contents such as signs, figures, or characters determined according to the measurement result of the wearable sensor 20 may be displayed.

Regarding information to be displayed together with the image of the participant J participating in the lesson, information other than the information corresponding to the number of heartbeat may be included. In one or more embodiments of the present invention, as shown in FIGS. 5 and 6, in addition to the information corresponding to the number of heartbeat, a text box Ty in which an attribute (personal information) of the participant J participating in the lesson or consumed calories after start of lesson participation are described is displayed as pop-up immediately above a region where a head portion of the participant J is presented. However, the present invention is not limited to this but it is also possible to further add any information useful for grasping a state (current state) of the participant J participating in the lesson.

### <<Configuration of Second Data Processing Terminal>>

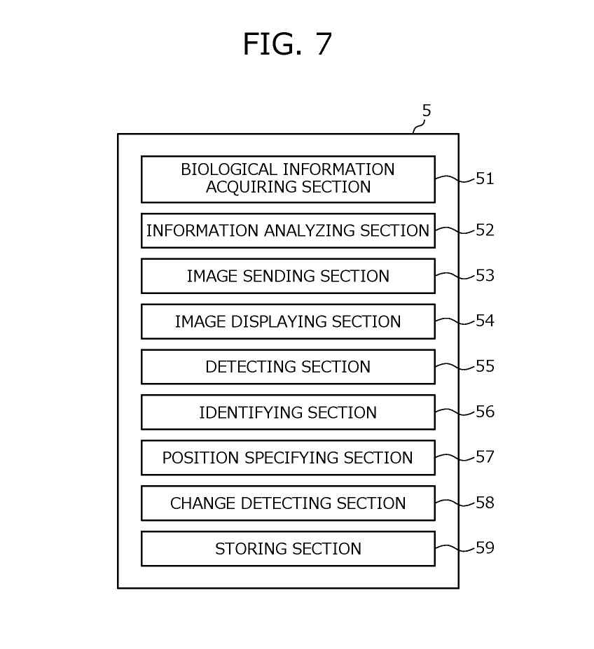

Next, a functional configuration of the second data processing terminal 5 will be newly described. The computer forming the second data processing terminal 5 functions as the image display device according to one or more embodiments of the present invention by executing the above second program. In other words, the second data processing terminal 5 includes plural functional sections, and specifically has a biological information acquiring section 51, an information analyzing section 52, an image sending section 53, an image displaying section 54, a detecting section 55, an identifying section 56, a position specifying section 57, a change detecting section 58, and a storing section 59 as shown in FIG. 7. These are formed by co-working of hardware devices forming the second data processing terminal 5 (specifically, the CPU, the memories, the communication interface, and the hard disk drive) and the second program. Hereinafter, the functional sections will be described.

(Biological Information Acquiring Section 51)

The biological information acquiring section 51 is to acquire the number of heartbeat of the participants J measured by the wearable sensors 20 at the preset time interval. Speaking in more detail, in normal times, the biological information acquiring section 51 acquires the number of heartbeat of each participant J stored in the biological information storage server 30 by communicating with the biological information storage server 30 at the first time interval t1. The number of heartbeat of each participant J acquired at this time is stored in the second data processing terminal 5 by each participant J.

In a case where there are participants J participating in the lesson in gym P1, the second data processing terminal 5 acquires the number of heartbeat of the participants J by communicating with the wearable sensors 20 worn by the participants J participating in the lesson. Speaking in more detail, when the detecting section 55 to be described later detects the participants J participating in the lesson and specifies identification information (participant IDs) of the participants J, the biological information acquiring section 51 cites a sensor ID storage table shown in FIG. 8, and specifies sensor IDs associated with the participant IDs specified by the detecting section 55. The sensor ID storage table is to regulate an association relationship between the participant IDs assigned to the participants J and the sensor IDs serving as identification information of the wearable sensors 20 worn by the participants J, and is stored in the storing section 59.

The biological information acquiring section 51 acquires the number of heartbeat of the participants J participating in the lesson by communicating with the wearable sensors 20 to which the specified sensor IDs are assigned. In one or more embodiments of the present invention, the biological information acquiring section 51 acquires the number of heartbeat of the participants J at the second time interval t2 while the participants J participating in the lesson are participating in the lesson (in other words, the detecting section 55 is detecting the participants J participating in the lesson).

(Information Analyzing Section 52)

The information analyzing section 52 is to add up the number of heartbeat of each participant J acquired at the first time interval t1 in normal times by the biological information acquiring section 51 for each participant J and to analyze the number of heartbeat for each participant J. More specifically speaking, the information analyzing section 52 averages the number of heartbeat of each participant J acquired at the first time interval t1 by each participant J to calculate the average number of heartbeat of each participant J. In one or more embodiments of the present invention, several dozen sets of the number of heartbeat acquired in the past are averaged to calculate the average number of heartbeat. However, a range of the number of heartbeat set as an object at the time of calculating the average number of heartbeat may be arbitrarily determined.

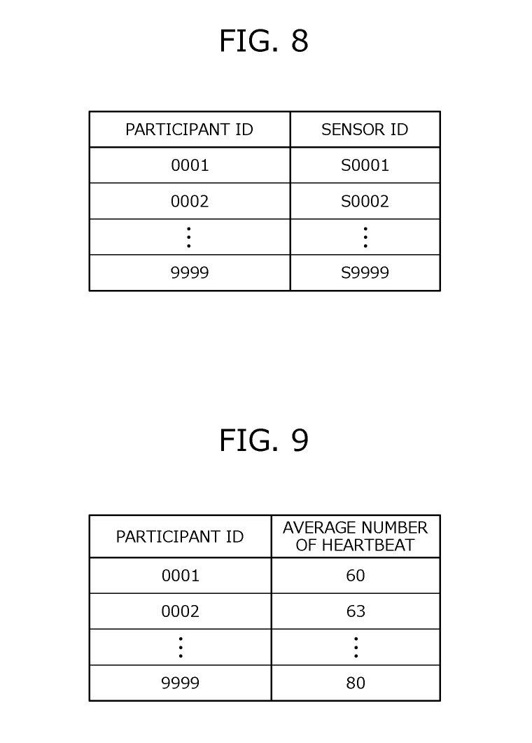

The average number of heartbeat for each participant J calculated by the information analyzing section 52 is stored in the storing section 59 in a state where the average number of heartbeat is associated with the participant ID, and specifically stored as an average number of heartbeat storage table shown in FIG. 9.

(Image Sending Section 53)

With performance of a predetermined action in the dedicated booth P2 by the instructor I (for example, the instructor standing at a position in front of the display 11 in the dedicated booth P2) as momentum, the image sending section 53 controls the camera 13 and the microphone 14 installed in the dedicated booth P2 so that the camera shoots an image and the microphone collects a sound in the dedicated booth P2. After that, the image sending section 53 embeds the sound collected by the microphone 14 into the image shot by the camera 13 and then sends the shot image toward the participant side unit 2. In one or more embodiments of the present invention, the image into which the sound is embedded is sent. However, the present invention is not limited to this but the image and the sound may be separately and individually sent.

(Image Displaying Section 54)

The image displaying section 54 displays the image received from the participant side unit 2, that is, the real-time image being shot by the camera 13 installed in the gym P1 on the display 11. The image displaying section 54 emits the reproduced sound at the time of reproducing the sound embedded in the received image through the speaker 12.

In a case where the change detecting section 58 to be described later detects the change in the face position of the instructor I, the image displaying section 54 shifts a range of the image received from the participant side unit 2, the range being displayed on the display 11 installed in the dedicated booth P2 according to the face position after the change. That is, when the face of the instructor I is laterally moved, the image displaying section 54 displays the image displaced from the currently displayed image by the amount corresponding to a moving distance of the face on the display 11.

Further, in a case where the figure image of the participant J participating in the lesson is included in the image received from the participant side unit 2, the image displaying section 54 displays, at the time of displaying the image, the information corresponding to the number of heartbeat of the participant J participating in the lesson while overlapping with the above image. More specifically speaking, the image displaying section 54 displays the text box Tx presenting the measurement result of the wearable sensor 20 worn by the participant J as pop-up in the region corresponding to the position of the figure image of the participant J participating in the lesson (strictly, the position specified by the position specifying section 57).

The image displaying section 54 updates display contents of the above text box Tx (that is, the number of heartbeat of the participant J participating in the lesson) every time the biological information acquiring section 51 acquires the number of heartbeat of the participant J participating in the lesson.

Further, in one or more embodiments of the present invention, the image displaying section 54 determines whether or not the number of heartbeat satisfies a preset condition upon displaying the number of heartbeat of the participant J participating in the lesson in the text box Tx. Specifically speaking, the image displaying section 54 determines whether or not a current value of the number of heartbeat exceeds a threshold value regarding the participant J participating in the lesson.

The “threshold value” is a value used as a condition at the time of determining whether or not there is an abnormality regarding the participant J participating in the lesson (specifically, whether or not exercise performed in the lesson should be stopped), and a threshold value is set for each participant J. In one or more embodiments of the present invention, the threshold value for each participant J is set according to the average number of heartbeat calculated by the information analyzing section 52 by each participant J. The set threshold value for each participant J is stored in the storing section 59. Further, the threshold value is set every time the average number of heartbeat is updated.

A specific method at the time of setting the threshold value according to the average number of heartbeat is not particularly limited. In one or more embodiments of the present invention, the threshold value is set according to the average number of heartbeat. However, the threshold value may be set according to parameters other than the average number of heartbeat (for example, the age, the sex, or biological information other than the average number of heartbeat). In one or more embodiments of the present invention, the threshold value is set for each participant J. However, the present invention is not limited to this but a single threshold value may be set and the threshold value may be used as a common threshold value for all the participants J.

At the time of displaying the number of heartbeat of the participant J participating in the lesson in the text box Tx, the image displaying section 54 displays in a display mode corresponding to the above determination result. Specifically, regarding the participant J whose current number of heartbeat does not exceed the threshold value, the number of heartbeat is displayed in a display mode in normal times. Meanwhile, regarding the participant J whose current number of heartbeat exceeds the threshold value, the number of heartbeat is displayed in a display mode for abnormality notification. In this way, by determining a magnitude relationship between the current value of the number of heartbeat and the threshold value and displaying the number of heartbeat in the display mode corresponding to the determination result, when the current value of the number of heartbeat becomes an abnormal value, it is possible to promptly notify the instructor I of the situation.

The “display mode” includes a color of text indicating the number of heartbeat, a background color of the text box Tx, size of the text box Tx, a shape of the text box Tx, blinking/non-blinking of the text box Tx, generation/non-generation of an alarming sound, etc.

(Detecting Section 55)

The detecting section 55 is to specify the participant J in a predetermined state, specifically, the participant J in a state of participating in the lesson in the gym P1. That is, the participant J participating in the lesson corresponds to the “specified subject” who is a subject to be detected by the detecting section 55.

A method of detecting the participant J participating in the lesson by the detecting section 55 will be described. Based on the image and the depth data received form the participant side unit 2, it is determined whether or not a figure image is included in the received image. In a case where the figure image is included, motion of the figure image (that is, motion of the person whose figure image is presented) is detected based on the figure image and the skeleton model. In a case where the detected motion is predetermined motion (specifically, motion having a degree of matching with an action of the instructor I is a fixed degree or more), the person whose figure image is presented is detected as the participant J participating in the lesson.

The method of detecting the participant J participating in the lesson is not limited to the above method. For example, by installing a position sensor in the gym P1, outputting a signal when such a position sensor detects the participant J standing at the position in front of the display 11 which is installed in the gym P1, and receiving the signal by the detecting section 55, the participant J participating in the lesson may be detected.

(Identifying Section 56)

The identifying section 56 is to recognize the participant J in a case where the detecting section 55 detects the participant J participating in the lesson. The identifying section 56 analyzes the figure image of the participant J when the detecting section 55 detects the participant J participating in the lesson. Specifically speaking, the identifying section 56 implements an image analysis of matching an image of a face part of the participant J participating in the lesson detected by the detecting section 55 with a face picture image of the participant J registered in advance. Thereby, the identifying section 56 specifies who is the participant J participating in the lesson. Further, the identifying section 56 specifies the identification information (participant ID) of the participant J participating in the lesson based on a specifying result.

(Position Specifying Section 57)

The position specifying section 57 is to specify, when the figure image of the participant J participating in the lesson is included in the image received from the participant side unit 2, a position of the figure image (strictly, a position in the image displayed on the display 11 which is installed in the dedicated booth P2). When receiving the image and the depth data received from the participant side unit 2, the position specifying section 57 specifies the position of the figure image of the participant J participating in the lesson in accordance with the procedure described above.

The position is sequentially specified by the position specifying section 57 throughout a period of the detecting section 55 detecting the participant J participating in the lesson. Therefore, when the position of the participant J participating in the lesson is moved due to exercise, the position specifying section 57 immediately specifies the position after movement (position of the figure image indicating the moved participant J).

(Change Detecting Section 58)

The change detecting section 58 is to detect, when the face position of the instructor I who is confirming the displayed image on the display 11 in the dedicated booth P2 is moved, the change in the position. When the change detecting section 58 detects the change in the face position of the instructor I, as described above, the range of the image received from the participant side unit 2, the range being displayed on the display 11 of the dedicated booth P2 by the image displaying section 54 is shifted according to the change in the face position of the instructor I. That is, the change detecting section 58 detects the change in the face position of the instructor I as momentum of starting the look-in processing described above.

In one or more embodiments of the present invention, the change detecting section 58 detects the change in the face position of the instructor I standing in front of the display 11. However, an object to be detected is not limited to the change in the face position but contents other than the face position, for example, a change in a facial direction or a line of sight of the instructor I may be detected. That is, the change detecting section 58 may detect the change in at least one of the face position, the facial direction, and the line of sight of the instructor I as momentum of starting the look-in processing.

(Storing Section 59)

The storing section 59 stores the sensor ID storage table shown in FIG. 8 and the average number of heartbeat storage table shown in FIG. 9. The storing section 59 stores the threshold value set for determining whether or not the number of heartbeat of the participant J participating in the lesson is an abnormal value by each participant J. In addition, the storing section 59 stores the personal information, an elapsed time after start of the lesson, consumed calories (specifically, the information displayed in the text box Ty shown in FIGS. 5 and 6) regarding the participant J.

### <<Flow of Exercise Instruction>>

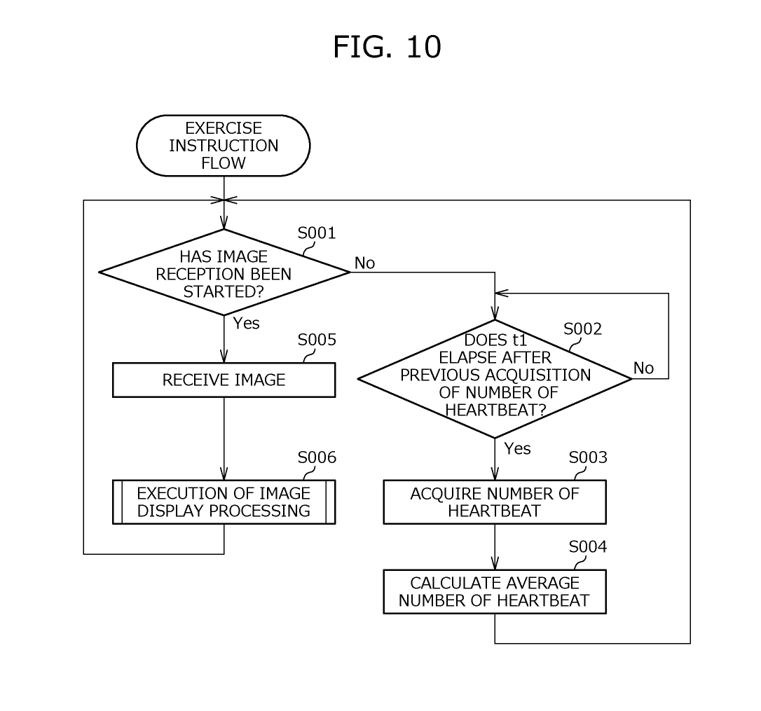

Next, a flow of exercise instruction using the exercise instruction system 1 will be described with reference to FIG. 10. In the exercise instruction flow to be described below, an image display method according to one or more embodiments of the present invention is adopted. That is, hereinafter, as description regarding the image display method according to one or more embodiments of the present invention, procedure of the exercise instruction flow to which the image display method is applied will be described. In other words, steps in the exercise instruction flow to be described below correspond to constituent elements of the image display method according to one or more embodiments of the present invention.

The exercise instruction flow is mainly divided into two flows as shown in FIG. 10. One of the flows is a flow of the time when the instructor side unit 3 receives the image from the participant side unit 2. The other flow is a flow of the time when the instructor side unit 3 does not receive the image from the participant side unit 2, that is, the flow in normal times. In the participant side unit 2, with the participant J performing a predetermined action in the gym P1 (for example, standing at the position in front of the display 11 in the gym P1) as a trigger, image shooting and sound collection are started in the gym P1, and the image is sent toward the instructor side unit 3.

First, the flow in normal times when the instructor side unit 3 does not receive the image from the participant side unit 2 (case of No in S001) will be described. When the instructor side unit 3 does not receive the image from the participant side unit 2, that is, when there is no participant J participating in the lesson in the gym P1, the computer provided in the instructor side unit 3 (that is, the second data processing terminal 5) regularly communicates with the biological information storage server 30 and acquires the number of heartbeat of each participant J.

More specifically speaking, the second data processing terminal 5 communicates with the biological information storage server 30 at the first time interval t1. In other words, at the time point when t1 elapses after the previous acquisition of the number of heartbeat (S002), the second data processing terminal 5 communicates with the biological information storage server 30 and acquires the number of heartbeat of each participant J, that is, the measurement result of the wearable sensor 20 (S003).

After acquiring the number of heartbeat of each participant J, the second data processing terminal 5 calculates the average number of heartbeat based on the number of heartbeat acquired at this time and the number of heartbeat acquired previously (S004). In this Step S004, the second data processing terminal 5 calculates the average number of heartbeat for each participant J. The second data processing terminal 5 stores the calculated average number of heartbeat for each participant J, specifically keeps in the average number of heartbeat storage table.

Next, a flow of the time when the instructor side unit 3 starts receiving the image (case of Yes in S001) will be described. When receiving the image sent from the participant side unit 2 (S005), the second data processing terminal 5 executes image display processing with this as a trigger (S006).

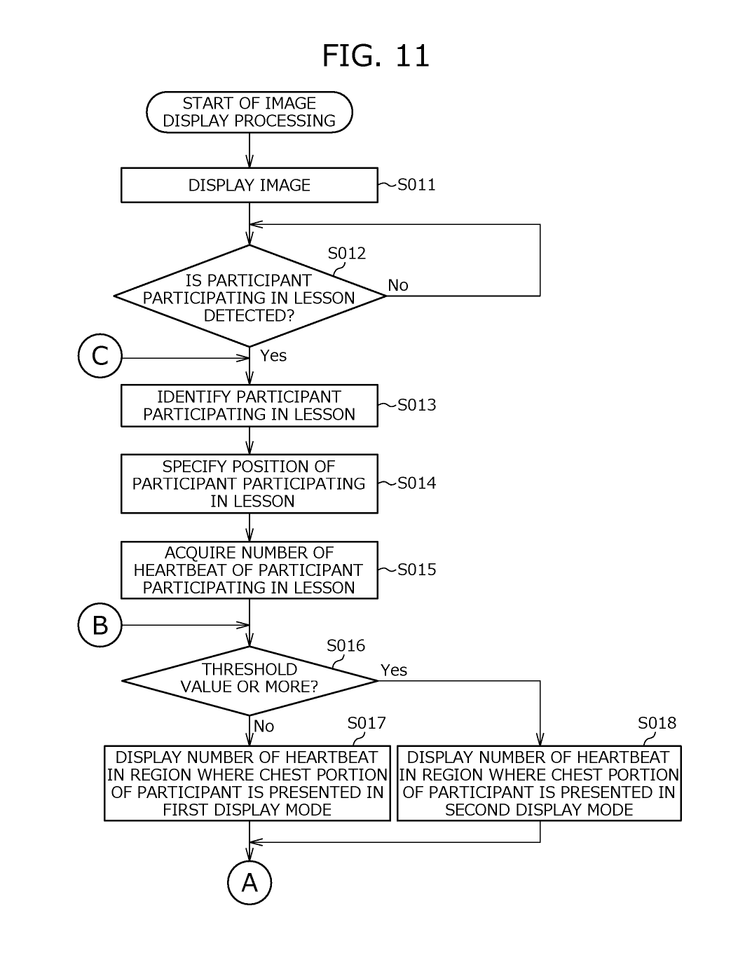

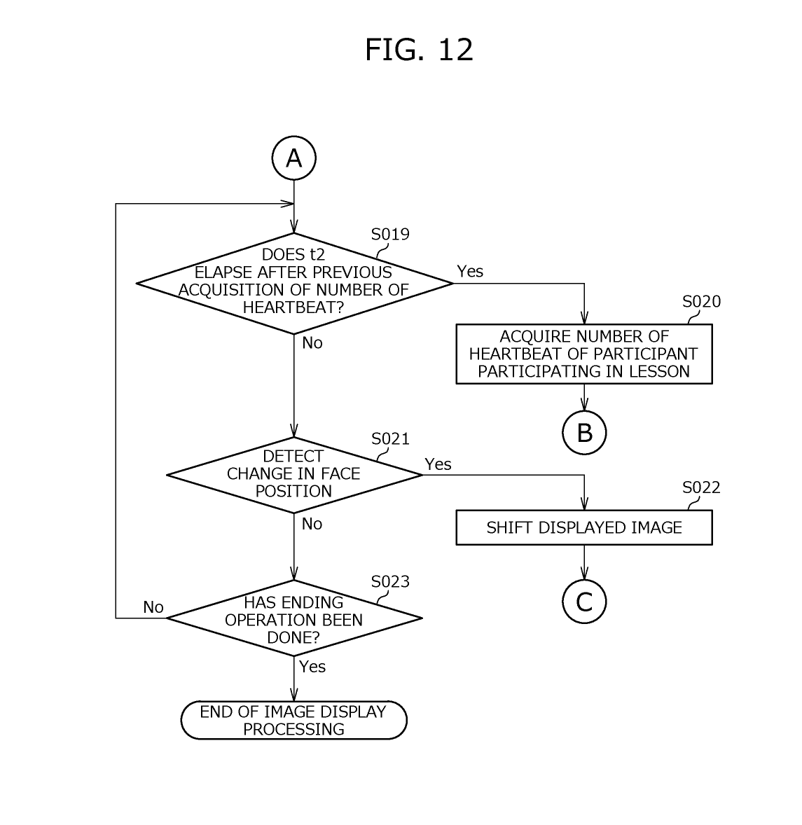

Hereinafter, a flow of the image display processing will be described with reference to FIGS. 11 and 12. In the image display processing, the second data processing terminal 5 first displays the image received from the instructor side unit 3 (that is, the image being shot by the camera 13 installed in the gym P1) on the display 11 installed in the dedicated booth P2 (5011). In one or more embodiments of the present invention, size of the received image is larger than screen size of the display 11. Thus, the second data processing terminal 5 displays part of the received image (partial image) on the display 11.

Next, the second data processing terminal 5 determines whether or not there is any participant J participating in the lesson in the gym P1 based on the image received from the participant side unit 2 and the depth data received together with the received image (S012). In a case of detecting the participant J participating in the lesson (that is, in a case of determining that there is a participant J participating in the lesson in the gym P1), the second data processing terminal 5 analyzes a figure image of the participant J to identify the participant J (S013). By this step, the second data processing terminal 5 specifies identification information (participant ID) of the identified participant J.

The second data processing terminal 5 specifies a position of the figure image of the participant J participating in the lesson in the displayed image (image displayed on the display 11) including the figure image (S014). In one or more embodiments of the present invention, based on the image and the depth data received from the participant side unit 2, and the skeleton model, the position of the figure image is specified by the method described above.

The second data processing terminal 5 reads the identification information (sensor ID) of the wearable sensor 20 associated with the participant J participating in the lesson who was identified in Step S013 out of the sensor ID storage table stored inside the second data processing terminal. After that, the second data processing terminal 5 acquires the number of heartbeat of the participant J participating in the lesson by communicating with the wearable sensor 20 specified by the read sensor ID (S015).

After acquiring the number of heartbeat of the participant J participating in the lesson, the second data processing terminal 5 reads the average number of heartbeat associated with the participant J participating in the lesson who was identified in Step S013 out of the above average number of heartbeat storage table. After that, the second data processing terminal 5 sets a threshold value according to the read average number of heartbeat, and determines a magnitude relationship between the threshold value and the number of heartbeat acquired in Step S015 (S016). The threshold value set in this Step S016 is a threshold value (condition) associated with the participant J participating in the lesson.

The second data processing terminal 5 displays the text box Tx presenting the number of heartbeat acquired in Step S015 in addition to the image including the figure image of the participant J participating in the lesson on the display 11 installed in the dedicated booth P2. At this time, the second data processing terminal 5 displays the above text box Tx in a region corresponding to the position specified in Step S014 in a display mode corresponding to a determination result in the previous Step S016 while overlapping with the above image.

More specifically speaking, in a case where the acquired number of heartbeat is lower than the threshold value (No in S016), the second data processing terminal 5 displays the above text box Tx as pop-up in the region of the display screen of the display 11 where the chest portion of the participant J participating in the lesson is presented in a first display mode (S017). On the other hand, in a case where the acquired number of heartbeat is more than the threshold value (Yes in S016), the second data processing terminal 5 displays the above text box Tx as pop-up in the region of the display screen of the display where the chest portion of the participant J participating in the lesson is presented in a second display mode (S018). The first display mode and the second display mode are display modes different from each other, and for example, indicate the background color of the text box Tx.

In one or more embodiments of the present invention, the second data processing terminal 5 acquires the number of heartbeat of the participant J participating in the lesson at the second time interval t2 shorter than the first time interval t1 while detecting the participant J participating in the lesson. In other words, while detecting the participant J participating in the lesson, the second data processing terminal 5 communicates with the wearable sensor 20 worn by the participant J participating in the lesson every time a time corresponding to t2 elapses (S019), and acquires the number of heartbeat of the participant J (S020).

The second data processing terminal 5 repeats Steps S016 to S018 and updates display contents of the text box Tx (that is, the current value of the number of heartbeat) every time the number of heartbeat is newly acquired. That is, while the participant J participating in the lesson is being detected, the number of heartbeat of the participant J participating in the lesson acquired at t2 by the second data processing terminal 5 is presented in the text box Tx displayed while overlapping with the image. At the time of updating the display contents of the text box Tx, the second data processing terminal 5 determines a magnitude relationship between the newly acquired number of heartbeat and the threshold value every time, and displays the above text box Tx in the display mode corresponding to a determination result.

Meanwhile, during a period from the previous acquisition of the number of heartbeat to the elapse of t2, when the instructor I staying in front of the display 11 laterally moves the face, the second data processing terminal 5 detects the change in the face position of the instructor I (S021). With this as a trigger, the second data processing terminal 5 executes the look-in processing. In such processing, the range of the image displayed on the display 11 in the dedicated booth P2 is shifted according to the change in the face of the instructor I (S022). Specifically, the range of the image received from the participant side unit 2, the range being displayed on the display 11 is displaced by an amount corresponding to the moving amount of the face in the direction opposite to the moving direction of the face of the instructor I.

In a case where the look-in processing is executed and the displayed image is shifted, the second data processing terminal 5 returns to Step S012 and identifies the participant J from the figure image of the participant J participating in the lesson included in the displayed image after shifting. After that, the second data processing terminal 5 repeatedly performs the following steps (steps including Step S013 and after) in the same procedure as the procedure described above.

Until the instructor I performs a predetermined ending operation (for example, an operation of moving away from the display 11 in the dedicated booth P2 by a predetermined distance) (S023), the second data processing terminal 5 repeatedly performs the series of steps described above. At the end, the second data processing terminal 5 ends the image display processing at the time point when the instructor I performs the above ending operation.

### <<Effectiveness of Embodiments>>

As described above, in one or more embodiments of the present invention, in a case where there is a participant J participating in the lesson in the gym P1, the image including the figure image of the participant J is displayed on the display 11 installed in the dedicated booth P2. At this time, information corresponding to the number of heartbeat of the participant J participating in the lesson (specifically, the text box Tx presenting the current value of the number of heartbeat) is displayed while overlapping with the image. In this way, by confirming the number of heartbeat together with the image regarding the participant J participating in the lesson, it is possible to monitor the state of the participant J (for example, the degree of understanding of the instructions, the fatigue degree, the adequacy of physical movement, etc.)

In one or more embodiments of the present invention, the above text box Tx is displayed in the region of the display screen of the display 11, the region corresponding to the position of the figure image of the participant J participating in the lesson. Therefore, for example, even when the position of the participant J participating in the lesson is moved in the lesson, the above text box Tx is, in conjunction with that motion, moved so as to maintain a relative positional relationship with the figure image of the participant J. Thereby, for example, in a situation where plural participants J are participating in the lesson, and even after the respective participants J move, it is possible to easily grasp whose number of heartbeat is presented in the text box Tx displayed on the display 11.

In one or more embodiments of the present invention, while the participant J participating in the lesson is being detected, the number of heartbeat of the participant J is acquired at the time interval shorter than in normal times (that is, at the second time interval t2). In this way, by making the time interval at which the number of heartbeat of the participant J participating in the lesson is acquired shorter than the time interval at which the number of heartbeat of the participant J not participating in the lesson is acquired (that is, the first time interval t1), when the number of heartbeat of the participant J participating in the lesson is changed, it is possible to more promptly grasp the change in the number of heartbeat. As a result, for example, in a case where the number of heartbeat of a certain participant J is radically increased during the lesson, the instructor I can quickly realize that situation.

Further, in one or more embodiments of the present invention, upon displaying the text box Tx presenting the number of heartbeat of the participant J participating in the lesson on the display 11, the magnitude relationship between the number of heartbeat and the threshold value is determined. The above text box Tx is displayed in the display mode corresponding to the determination result. Thereby, when the number of heartbeat of the participant J participating in the lesson is the threshold value or more (that is, when the number of heartbeat is abnormally high), it is possible to more easily remind the instructor I of such a situation.

In the aforementioned embodiments, an association relationship between the wearable sensor 20 and the participant J is determined in advance, and specifically, the above association relationship is regulated as the sensor ID storage table shown in FIG. 8. Therefore, in the aforementioned embodiments, in a case where information of the number of heartbeat is acquired from a certain wearable sensor 20, it is possible to specify which participant J's number of heartbeat it is by citing the above sensor ID storage table. In the aforementioned embodiments, when the figure image of the participant J is included in the image received from the participant side unit 2, whose image the figure image is is specified by an image identifying function (strictly, a face picture image identifying function) by the identifying section 56.

As described above, in the aforementioned embodiments, by specifying whose number of heartbeat the number of heartbeat acquired from the wearable sensor 20 is, and specifying whose image the figure image presented in the received image is, it is possible to associate the number of heartbeat and the figure image of the same participant J with each other. As a result, the number of heartbeat and the figure image associated with each other are displayed so that the association relationship thereof is clear. More specifically speaking, information corresponding to the number of heartbeat of a certain participant J is displayed in a region corresponding to a position of a figure image of the same participant J.

Meanwhile, it is considered that there is a case where the association relationship between the wearable sensor 20 and the participant J is not determined in advance unlike the aforementioned embodiments. For example, in a case where the wearable sensors 20 can be exchanged between the participants J or a case where a wearer of the wearable sensor 20 is changed in each lesson, it is difficult to grasp the association relationship between the wearable sensor 20 and the participant J in advance. In the aforementioned embodiments, whose image the figure image in the image is is specified by image identification processing. However, in this case, there is a possibility that the person to be specified is changed by precision of image identification. For example, in a case where size of the figure image is not sufficient size for performing the image identification processing, there is a possibility that precise specifying is not done.

When the association relationship between the figure image and the number of heartbeat is not properly grasped, it is difficult for the person who confirms both the figure image and the number of heartbeat (specifically, the instructor I) to accurately grasp the situation of each participant J. In particular, in a case where plural participants J are participating in the lesson, the above matter more remarkably occurs.

Thus, hereinafter, a case according to one or more embodiments of the present invention where the number of heartbeat and the figure image of each participant J are associated with each other by procedure different from the aforementioned embodiments will be described. One or more embodiments are common to the aforementioned embodiments excluding a method of associating the figure image and the number of heartbeat of each participant J. Hereinafter, only contents different from the aforementioned embodiments will be described. Hereinafter, for easy understanding of description, a case where three participants J (specifically, A, B, and C) participate in the lesson will be described as an example.

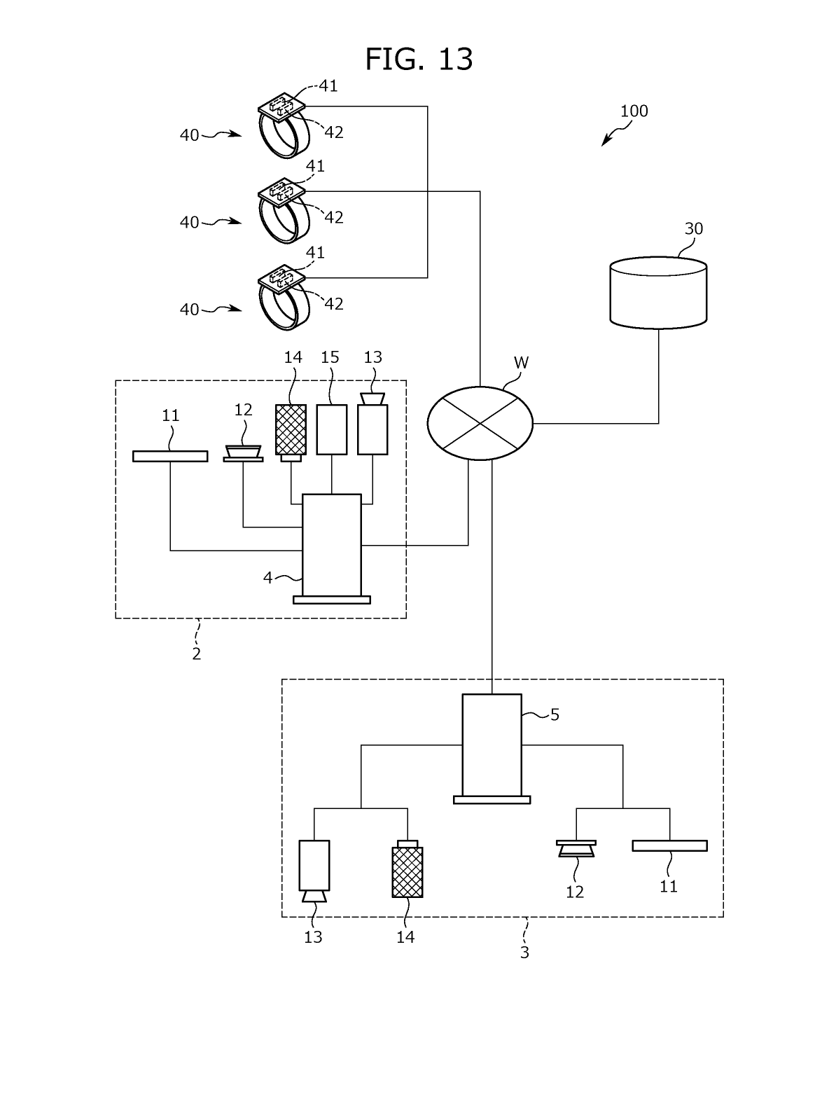

First, a communication system for exercise instruction (hereinafter, referred to as the exercise instruction system 100) according to one or more embodiments of the present invention will be described with reference to FIG. 13. As shown in FIG. 13, in the exercise instruction system 100 according to one or more embodiments of the present invention, a smart band 40 is used in place of the wearable sensor 20. This smart band 40 is a wristband type transmitter prepared for each participant J.

The smart band 40 will be described in detail. Each participant J wears the smart band 40 on the wrist at the time of participating in the lesson, and for example, wears a few days before the lesson day. The smart band 40 includes a heartbeat sensor 41 and an acceleration sensor 42, and sends respective measurement results of these sensors toward the second data processing terminal 5. The heartbeat sensor 41 is an example of the sensor, and regularly measures heartbeats of the participant J who is the wearer as well as the wearable sensor 20.

The acceleration sensor 42 is an example of the action detector, and detects an action of the participant J (strictly, an action of moving a hand on the side where the smart band 40 is worn) and generates action information as a detection result. This action information indicates information generated at the time of detecting the action of the participant J, the information corresponding to a degree of the action (for example, a moving amount of the hand). When the participant J performs a predetermined action (specifically, a response action to be described later), the acceleration sensor 42 generates action information different from the action information in normal times. Although the acceleration sensor 42 is used as the action detector in one or more embodiments of the present invention, it is possible to utilize any device that detects the action of the participant J and outputs information corresponding to the action as the action detector.

Further, the smart band 40 acquires the personal information of the participant J who is the wearer, specifically, a name of the participant J, and sends the name together with the respective measurement results of the heartbeat sensor 41 and the acceleration sensor 42 toward the second data processing terminal 5. In one or more embodiments of the present invention, upon acquiring the name of the participant J, the smart band 40 communicates with a mobile terminal (not shown) held by the participant J such as a smartphone. Thereby, the smart band 40 acquires the name of the participant J stored in the mobile terminal. However, the method of acquiring the name is not particularly limited. For example, the smart band 40 may include an input means (not shown) and the person wearing the smart band 40 may operate the input means by himself/herself to input his/her name.

The information sent together with the number of heartbeat by the smart band 40, that is, the action information generated by the acceleration sensor 42 detecting the action of the participant J, and the name of the participant J acquired by the smart band 40 correspond to the “other information” regarding the participant J. The information sent from the smart band 40 is not limited to the above information but may further include information other than the above information.

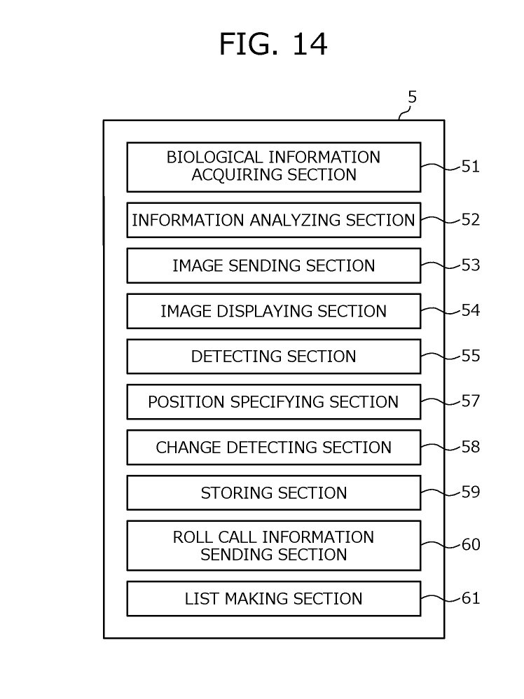

Next, with reference to FIG. 14, contents of the functions of the second data processing terminal 5, the contents being peculiar to one or more embodiments of the present invention will be described. In one or more embodiments of the present invention, as shown in FIG. 14, the second data processing terminal 5 does not include the identifying section 56 but has a roll call information sending section 60 and a list making section 61. The roll call information sending section 60 corresponds to the control information sending section, and generates and sends roll call information as control information toward the first data processing terminal 4. When receiving the roll call information, the first data processing terminal 4 analyzes the information and controls the speaker 12 of the participant side unit 2, that is, the speaker 12 (device) installed in a place where there are plural participants J in accordance with an analysis result. Specifically speaking, the first data processing terminal 4 specifies a name of a single participant J from the roll call information and controls the speaker 12 to emit a sound indicating the name.

The list making section 61 makes a list of participant LJ to be described later. This list of participant LJ is cited when the above roll call information sending section 60 generates the roll call information.

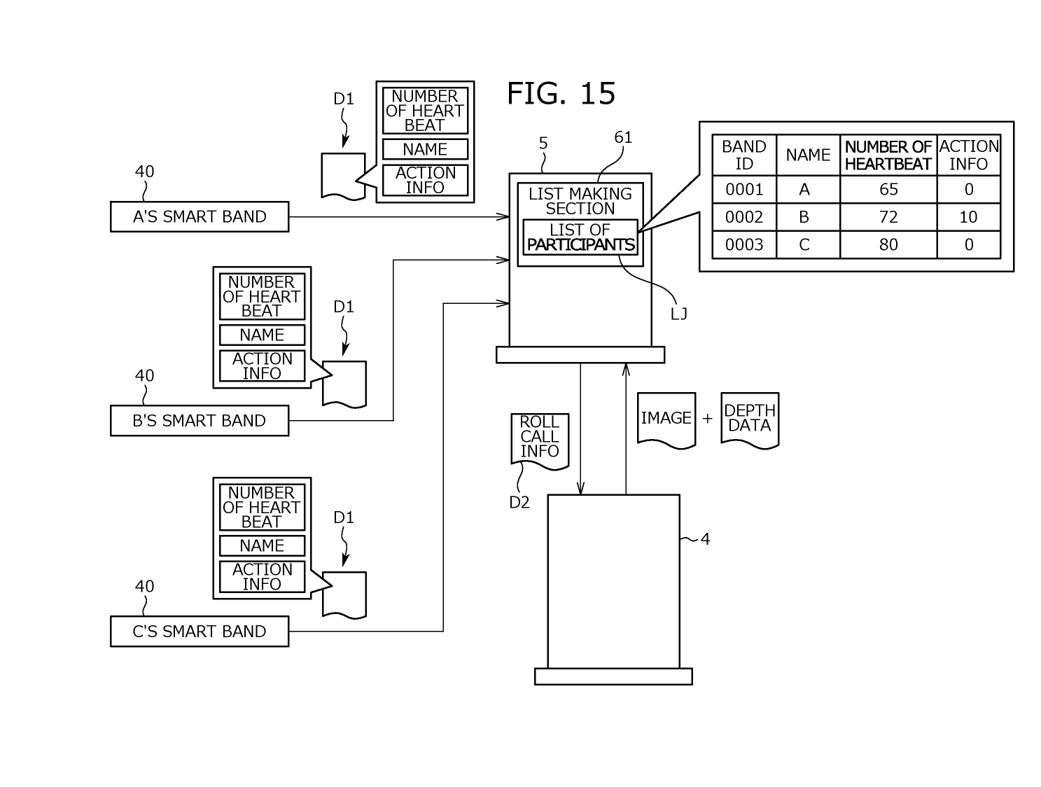

Next, the functions of the second data processing terminal 5 in one or more embodiments of the present invention will be described with reference to FIG. 15. FIG. 15 schematically shows exchanges of information centering the second data processing terminal 5. In one or more embodiments of the present invention, the smart band 40 worn by each participant J transmits transmitted information D1 as shown in FIG. 15. This transmitted information D1 is information including the number of heartbeat measured by the heartbeat sensor 41, the action information generated by the acceleration sensor 42 detecting the motion of each participant J, and the name of each participant J.

The biological information acquiring section 51 of the second data processing terminal 5 acquires the number of heartbeat, the name, and the action information of each participant J by receiving the transmitted information D1 from the smart band 40 of each participant J. As a result, the second data processing terminal 5 is notified of the number of heartbeat, the name, and the action information by each participant J. Meanwhile, the list making section 61 of the second data processing terminal 5 makes the list of participant LJ based on the transmitted information D1 acquired for each participant J. The list of participant LJ will be described. As shown in FIG. 15, a band ID serving as identification information of each smart band 40, and the name, the number of heartbeat, and the action information of the participant J sent from the smart band 40 are collected in the list of participant LJ in a table form.

In the case shown in FIG. 15, the list of participant LJ showing names, etc. of three participants J (A, B, and C) is made. Once the list of participant LJ is made, the roll call information sending section 60 of the second data processing terminal 5 cites the list of participant LJ, and specifies the name of a single participant J among the names of plural participants J listed. Then, the roll call information sending section 60 generates and sends roll call information D2 to call the name of the specified single participant J toward the first data processing terminal 4.

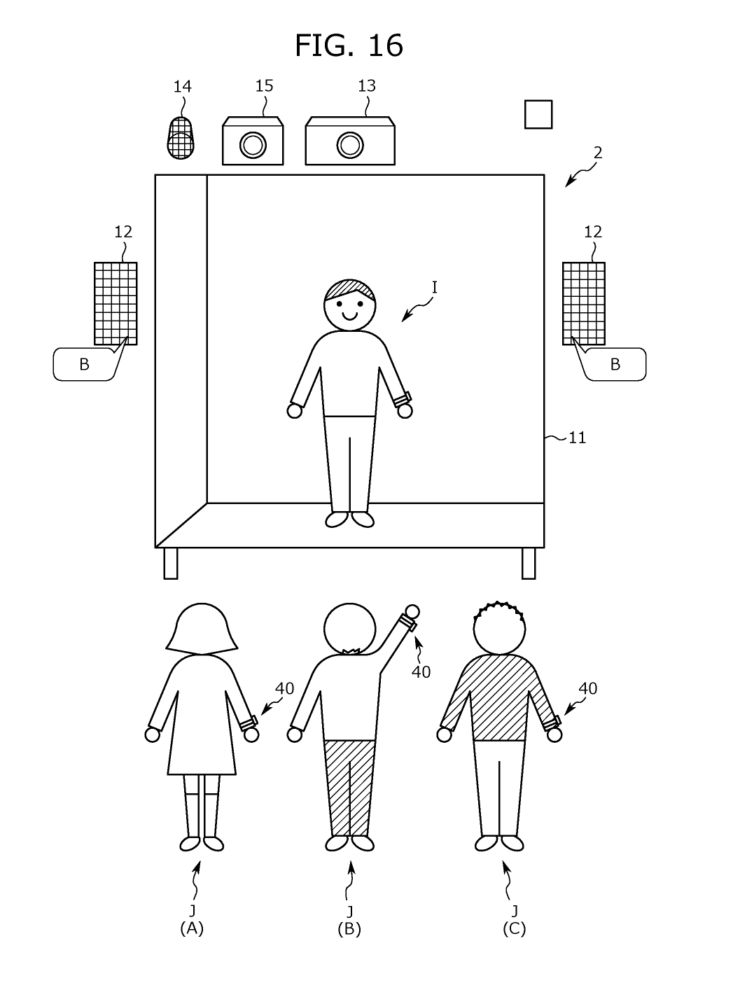

When receiving the roll call information D2, the first data processing terminal 4 specifies the name of the participant J indicated by the roll call information D2, and then controls the speaker 12 so that a sound indicating the specified name of the participant J is generated. Thereby, among the participants J staying in front of the speaker 12, the participant J whose name is called (hereinafter, referred to as the subject participant) is to perform a response action reacting to the sound. Specifically speaking, when a sound indicating his/her name emitted from the speaker 12 as shown in FIG. 16, the subject participant is to perform an action of raising his/her hand on the side of wearing the smart band 40 to respond. FIG. 16 is a view showing a state where one of the plural participants J (B in the case shown in the figure) is performing the response action.

As described above, by controlling the speaker 12 in accordance with the roll call information D2, a single participant J (subject participant) is encouraged to perform the above response action. In this sense, it can be said that the roll call information D2 is control information for controlling the speaker 12 of the participant side unit 2 so as to encourage one of the plural participants J to perform the response action.

Meanwhile, as the subject participant is performing the above response action, the camera 13 of the participant side unit 2 shoots an image including a figure image of the subject participant, and the microphone 14 collects a sound including a voice of the subject participant (specifically, a voice of the time of responding to the sound emitted from the speaker 12). The infrared sensor 15 of the participant side unit 2 measures the depth of the above image by predetermined pixel, and the first data processing terminal 4 acquires the depth data of the above image based on the measurement result of the infrared sensor 15. The first data processing terminal 4 sends the image on which the sound collected by the microphone 14 is superimposed, and the depth data toward the participant side unit 2. The second data processing terminal 5 receives the image and the depth data sent from the participant side unit 2.

In one or more embodiments of the present invention, when the second data processing terminal 5 receives the above image and the depth data, the position specifying section 57 executes processing of specifying a position of the figure image of the subject participant in the received image (hereinafter, referred to as the position specifying processing). The position specifying processing corresponds to the “first processing” according to one or more embodiments of the present invention. In this position specifying processing, the position of the figure image of the participant J who has performed the response action (that is, the subject participant), the position within the received image (hereinafter, simply referred to as the “position”) is specified.

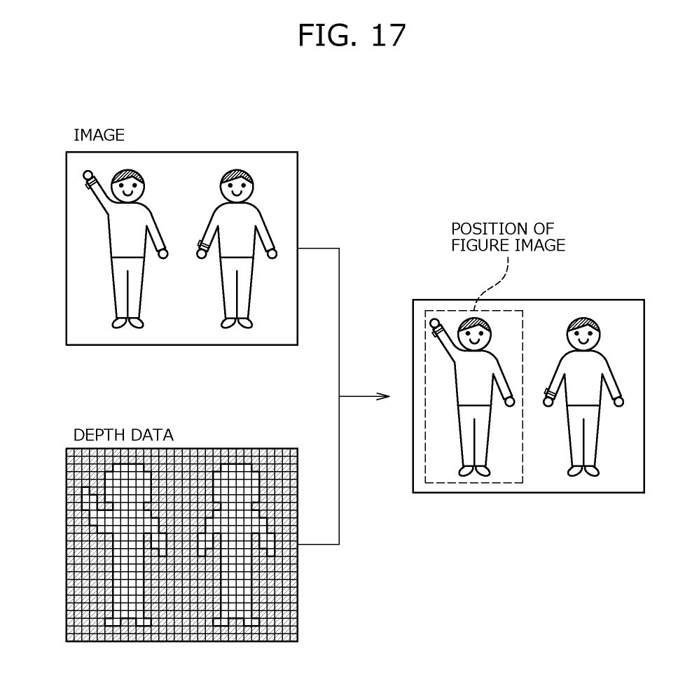

The position specifying section 57 according one or more embodiments of the present invention specifies the position of the figure image of the subject participant based on the depth data received together with the image by the second data processing terminal 5 in the position specifying processing. Speaking with reference to FIG. 17, the position specifying section 57 extracts pixels of the figure image from the pixels constituting the depth data in accordance with the same procedure as the above procedure. At this time, the position specifying section 57 applies a skeleton model of a human being, strictly, a skeleton model of a person who is performing an action of raising one hand. Thereby, the position specifying section 57 extracts pixels associated with the above skeleton model among the pixels constituting the depth data, that is, the pixels associated with the figure image of the subject participant who has performed the response action. In the depth data shown in FIG. 17, there are two white pixel groups (that is, pixel groups of the figure image), and among the pixel groups, the pixel group placed on the left side corresponds to the pixels of the figure image of the subject participant.

The position specifying section 57 specifies an image associated with the pixels in the received image (image shot by the camera 13 of the participant side unit 2) based on the pixels of the subject participant extracted from the depth data, and this image serves as the figure image of the subject participant. As a result, the position of the figure image of the subject participant is specified.

The method of specifying the position of the figure image of the subject participant is not limited to the method of specifying based on the depth data as described above. For example, the position of the figure image of the subject participant may be specified by analyzing the sound reproduced together with the image. Specifically speaking, the sound including the voice generated by the subject participant at the time of the response action is embedded into the image including the figure image of the subject participant. The position of the figure image may be specified by specifying a position where the voice is generated by analyzing this sound, and catching the figure image displayed at the closest position to the position where the voice is generated as the figure image of the subject participant. The position of the figure image of the subject participant may also be specified by analyzing a predetermined part in the figure image included in the image. Specifically speaking, when the subject participant produces a voice to respond at the time of the response action, a mouth part is moved among the figure image of the subject participant. Thus, the position of the figure image may be specified by catching the figure image in which mouth motion is recognized as the figure image of the subject participant.

At the time point when the subject participant performs the above response action, the biological information acquiring section 51 acquires the transmitted information D1 once again from the smart band 40 of each participant J. At this time, the action information included in the transmitted information D1 which is acquired from the smart band 40 of the subject participant by the biological information acquiring section 51 has contents different from the action information in normal times. Specifically, the action information has contents (numerical value) outputted only when the acceleration sensor 42 detects the response action.

The position specifying section 57 executes processing of specifying the smart band 40 of the subject participant among the smart bands 40 provided for each participant J based on the transmitted information D1 acquired by the biological information acquiring section 51 (hereinafter, referred to as the band specifying processing). The band specifying processing corresponds to the “second processing” according to one or more embodiments of the present invention. In the band specifying processing, the smart band 40 that sent the transmitted information D1 including the action information generated when the acceleration sensor 42 detects the response action (specifically, the information outputted only when the acceleration sensor 42 detects the response action) is specified as the smart band 40 of the subject participant.

As described above, the position of the figure image of the subject participant and the smart band 40 of the subject participant are specified. As a result, the figure image of the subject participant and the number of heartbeat serving as the biological information are associated with each other. More specifically speaking, in one or more embodiments of the present invention, when plural participants J are detected by the detecting section 55, by making one of the participants J (that is, the subject participant) perform a predetermined action (specifically, the response action), the participant J who has performed the action is specified (determined) from viewpoints of both the image and the action information. Thereby, the figure image of the specified participant J is associated with the number of heartbeat sent from the same smart band 40 as of the action information of the specified participant J.

By the position specifying section 57 repeating the above procedure (that is, the position specifying processing and the band specifying processing) for each participant J, the figure image and the number of heartbeat are associated with each other for all the participants J.

Further, as a result of associating the figure image and the number of heartbeat with each other, the position where the information corresponding to the number of heartbeat is displayed is decided by a relationship between the number of heartbeat and the position of the associated figure image. Specifically speaking, information corresponding to the number of heartbeat of a certain participant J (for example, B) is displayed in a region corresponding to the position of the figure image associated with the number of heartbeat (that is, the figure image of B), in detail, in a region where a chest portion of the participant J (B) is presented.

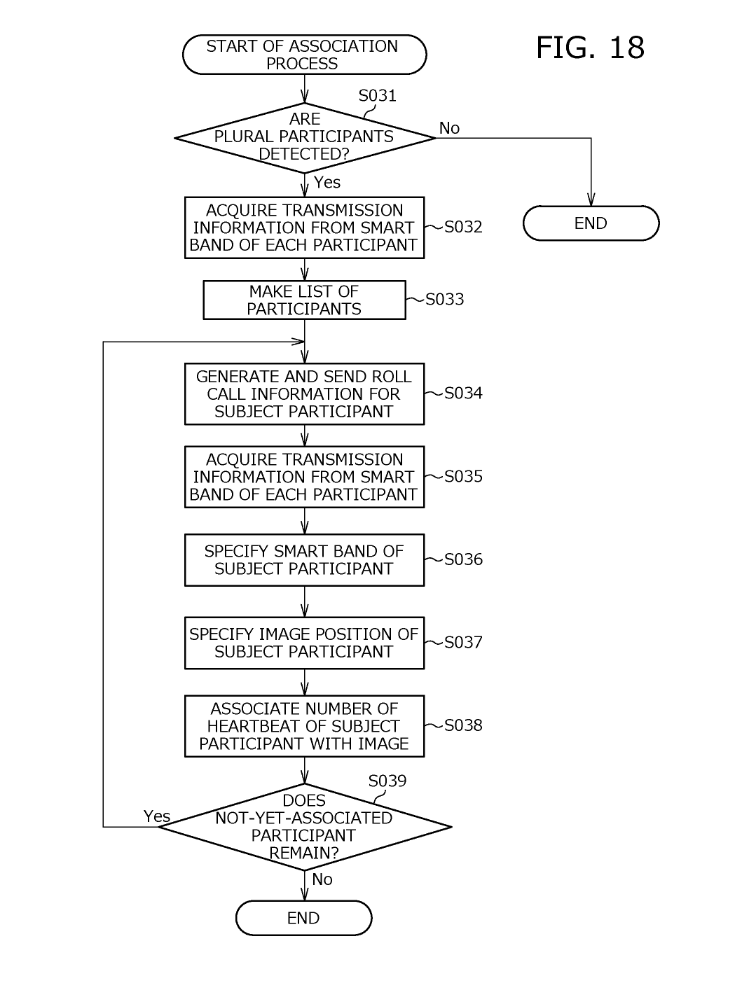

Next, a flow of a process of associating the number of heartbeat and the figure image of each participant J with each other (hereinafter, referred to as the association process) will be described with reference to FIG. 18. The association process is implemented by the second data processing terminal 5 in the already-described image display processing. More specifically speaking, in one or more embodiments of the present invention, the association process is implemented instead of Step S013 of identifying the participant J participating in the lesson and Step S014 of specifying the position of the participant J participating in the lesson in the steps of the image display processing shown in FIG. 11. The association process may be implemented only once in one image display processing, or may be implemented every time the instructor I moves the face position and the displayed image of the display 11 is shifted.

In the association process, first, it is determined whether or not plural participants J are detected by the detecting section 55 in the gym P1 (S031). In a case where plural participants J are detected in this Step S031, the association process is continued. In a case where plural participants J are not detected (that is, in a case where there is only one participant J), the association process is finished.

After Step S031, the biological information acquiring section 51 of the second data processing terminal 5 acquires the transmitted information D1 from the respective smart bands 40 of the plural participants J (S032). Thereby, the identification ID (band ID) of the smart band 40 worn by each participant J, and the name, the number of heartbeat, and the action information of each participant J are acquired by each participant J.

After that, the list making section 61 of the second data processing terminal 5 makes the list of participant LJ based on the transmitted information D1 acquired in the previous Step S032 (S033). After making the list of participant LJ, the roll call information sending section 60 of the second data processing terminal 5 cites the list of participant LJ and selects one of the plural participants J (subject participant), generates the roll call information D2 for the subject participant, and sends the information D2 toward the first data processing terminal 4 (S034). This Step S034 will be described in detail. The name of the subject participant is specified from the list of participant LJ, and the above roll call information D2 is generated as the control information for generating the sound to call the name.

Meanwhile, when receiving the roll call information D2, the first data processing terminal 4 specifies the name of the subject participant indicated by the roll call information D2, and controls the speaker 12 of the participant side unit 2 so that the sound indicating the name is emitted. Thereby, the sound to call the name of the subject participant is emitted from the speaker 12 in the gym Pl. The participant J corresponding to the subject participant performs the response action to the sound, specifically, raises a hand on the side of wearing the smart band 40 and also produces a voice to respond. Accordingly, the acceleration sensor 42 mounted on the smart band 40 which is worn by the subject participant detects the response action of the subject participant and generates the action information corresponding to a detection result.

Soon after sending the roll call information D2, the second data processing terminal 5 acquires the transmitted information D1 once again from the smart band 40 of each participant J (S035). Among the transmitted information D1 acquired at this time point, in the transmitted information D1 sent from the smart band 40 of the subject participant (that is, the participant J who has performed the response action), the action information generated by the acceleration sensor 42 detecting the response action is included.

The position specifying section 57 of the second data processing terminal 5 executes the band specifying processing, and specifies the smart band 40 of the subject participant based on the transmitted information D1 acquired in the previous Step S035 (S036). More specifically speaking, in the band specifying processing, the transmitted information D1 including the action information generated by the acceleration sensor 42 detecting the response action is specified, and then the smart band 40 serving as a source of the transmitted information D1 is specified as the smart band 40 of the subject participant.

During a period in which the association process is implemented, the second data processing terminal 5 receives the image in the gym P1 and the depth data of the image sent from the first data processing terminal 4. The position specifying section 57 of the second data processing terminal 5 executes the position specifying processing and specifies the position of the figure image of the subject participant (S037). More specifically speaking, among the depth data received together with the image, the pixels associated with the figure image of the subject participant who has performed the response action are extracted, and then the figure image associated with the extracted pixels (that is, the figure image of the subject participant) is specified in the received image, so that the position of the figure image is specified.

In one or more embodiments of the present invention, for the purpose of enhancing precision of specifying the position, in addition to specifying of the position of the figure image of the subject participant by the above method, the position of the figure image of the subject participant is separately specified by a second method and a third method. The second method is a method of specifying the position where the voice of the subject participant at the time of generating the response action by analyzing sound information embedded in the received image, and specifying the position of the figure image by catching the figure image displayed at the closest position to the specified position where the voice is generated as the figure image of the subject participant. The third method is a method of specifying the position of the figure image by catching the figure image of the participant J whose mouth part is moved for responding at the time of the response action as the figure image of the subject participant by analyzing the image of the mount part of each participant J included in the received image.

As described above, in one or more embodiments of the present invention, the position of the figure image of the subject participant is specified by the above three types of specifying methods. However, the present invention is not limited to this but the position of the figure image of the subject participant may be specified by adopting at least one of the above three types of specifying methods. The position of the figure image of the subject participant may also be specified by a method other than the above specifying methods.

The position specifying section 57 of the second data processing terminal 5 specifies the position of the figure image of the subject participant and the smart band 40, and as a result, associates the figure image of the subject participant with the number of heartbeat serving as the biological information (S038). That is, the position specifying section 57 associates the figure image of one of the plural participants J with the number of heartbeat sent from the smart band 40 which is worn by the person.

The steps after Step S032 described above are performed on all the plural participants J staying in the gym P1. That is, as long as the participant J whose figure image and the number of heartbeat are not yet associated with each other remains among the plural participants J staying in the gym P1, Steps 5032 to 038 described above are repeatedly implemented (S039). Thereby, the figure image and the number of heartbeat are associated with each other successively for each participant J staying in the gym P1. At the end, the association process is finished at the time point when the figure image and the number of heartbeat are associated with each other for all the plural participants J staying in the gym P1.

By implementing the association process described above, in one or more embodiments of the present invention, it is possible to precisely specify, regarding the number of heartbeat sent respectively from the smart bands 40 prepared for each participant J, which participant J's number of heartbeat it is among the plural participants J whose figure images are presented in the received image. In one or more embodiments of the present invention, upon realizing the above effect, there is no need for deciding the association relationship between the smart band 40 and the participant J in advance unlike the aforementioned embodiments, and there is also no need for identifying the participant J from the face picture image of the participant J. That is, in one or more embodiments of the present invention, it is possible to flexibly deal with even a case where the association relationship between the smart band 40 and the participant J is changed. In one or more embodiments of the present invention, when the number of heartbeat is acquired from a smart band 40 of a certain participant J (for example, B), it is possible to precisely find a figure image of the participant J (figure image of B) from the received image without using face picture image identification.

Further, in one or more embodiments of the present invention, by associating the number of heartbeat and the figure image with each other, the position where the information corresponding to the number of heartbeat is displayed is decided by the relationship between the number of heartbeat and the position of the associated figure image. As a result, during the image display processing, at the time of displaying the information corresponding to the number of heartbeat by each participant J in the steps after the association process, the information corresponding to the number of heartbeat is displayed in the region corresponding to the position of the figure image associated with the number of heartbeat. Thereby, the instructor I can confirm each of the plural participants J participating in the lesson while associating the figure images indicating the current state of the participants J and the current number of heartbeat with each other.

In one or more embodiments of the present invention, each participant J performs the response action to the sound to call his/her name as the predetermined action, and specifically performs the action of raising his/her hand to respond. In one or more embodiments of the present invention, with the response action as momentum, the figure image and the number of heartbeat of the participant J who has performed the response action (that is, the subject participant) are associated with each other. However, the action serving as momentum to associate the figure image and the number of heartbeat with each other is not particularly limited but may be an action other than the above response action.

In one or more embodiments of the present invention, in order to encourage the above response action, the second data processing terminal 5 generates and sends the roll call information D2 serving as the control information toward the first data processing terminal 4, and the first data processing terminal 4 controls the speaker 12 based on the roll call information D2. By the first data processing terminal 4 controlling the speaker 12, the sound indicating the name of the subject participant is emitted to encourage the above response action. However, the processing of encouraging the response action is not limited to the case where the processing is performed through the first data processing terminal 4 and the speaker 12 but for example performed by the instructor I. That is, by the instructor I citing the list of participant LJ and successively calling the names of the participants J on the list, it is possible to encourage the response action as well as the above embodiments. In this case, the instructor I selects the participants J one by one from the list of participant LJ, and in each case, inputs a selection result. The second data processing terminal 5 receives an input operation of the instructor I and specifies the selected participant J.

In one or more embodiments of the present invention, at the time of specifying the smart band 40 of the subject participant (participant J who has performed the response action), the smart band is specified with the action information outputted by the acceleration sensor 42 which is mounted on the smart band 40 as a clue. That is, when the participant J performs the response action, the acceleration sensor 42 of the smart band 40 worn by the person detects the response action, and outputs the action information corresponding to the response action. In the above embodiments, the smart band 40 of the subject participant is specified based on this action information. However, the method of specifying the smart band 40 of the subject participant is not limited to the above method but a method of specifying without using the action information outputted from the acceleration sensor 42 is also considered. Hereinafter, a case (modified example) where the smart band 40 of the subject participant is specified without using the action information will be described.

In the modified example, the identification ID (band ID) of the smart band 40 and the name and the number of heartbeat of each participant J are included in the transmitted information D1 transmitted from the smart band 40 of each participant J, whereas the action information is not included. In the modified example, as well as the above embodiments, when receiving the transmitted information D1 from the smart band 40 of each participant J, the second data processing terminal 5 makes the list of participant LJ, selects one of the participants J on the list, and generates the roll call information D2 to call the name of the selected participant J.

After the second data processing terminal 5 sends the roll call information D2, the first data processing terminal 4 receives the roll call information D2, specifies the name of the participant J indicated by the roll call information D2, and controls the speaker 12 so that the sound indicating the specified name is emitted. The participant J whose name is called in this control (that is, the subject participant) performs the response action. After that, the second data processing terminal 5 specifies the position of the figure image of the subject participant based on the image at the time point when the subject participant performs the response action and the depth data of the image. Thereby, the figure image and the name of the subject participant are associated with each other. Meanwhile, the association relationship between the name and the smart band 40 of the participant J is regulated by the list of participant LJ As above, the figure image and the smart band 40 of the subject participant are associated with each other, and as a result, the figure image and the number of heartbeat of the subject participant are associated with each other.

By the above procedure, in the modified example, it is possible to specify the smart band 40 of the subject participant without using the action information. As a result, it is possible to build up a system of a simpler configuration (exercise instruction system 100).

The image display device and the image display method according to one or more embodiments of the present invention are described above with an example. The above embodiments are mere examples and other examples are also considered. Specifically speaking, in the above embodiments, at the time of acquiring the number of heartbeat of the participant J participating in the lesson, by directly communicating with the wearable sensor 20 or the smart band 40 worn by the participant J, the number of heartbeat of the participant J participating in the lesson is acquired. However, the present invention is not limited to this. The number of heartbeat of the participant J participating in the lesson may be not acquired by directly receiving from the wearable sensor 20 or the smart band 40 but acquired from the biological information storage server 30 by communicating with the biological information storage server 30.

In the above embodiments, upon displaying the text box Tx presenting the number of heartbeat of the participant J participating in the lesson, the magnitude relationship between the current value of the number of heartbeat and the threshold value is determined and the above text box Tx is displayed in the display mode corresponding to the determination result. However, the contents of determination performed on deciding the display mode are not limited to the above contents. For example, by calculating a change ratio (change speed) of the number of heartbeat while deciding a standard value of the change speed in advance, a magnitude relationship between a calculation result of the change speed and the standard value may be determined.

In the above embodiments, when the current value of the number of heartbeat of the participant J participating in the lesson becomes the threshold value or more, in order to notify the instructor I of the fact that the current number of heartbeat is an abnormal value, the above text box Tx is displayed in the display mode different from the normal display mode. However, the method of notifying the instructor I of the abnormal state is not limited to the above method. For example, a message saying that the participant J is in an abnormal state may be displayed on the display 11. Alternatively, an alarm sound may be emitted from the speaker 12 installed in the dedicated booth P2.

In the above embodiments, the participant J participating in the lesson in the gym P1 (strictly, the participant J making the same motion as the instructor I) is detected as the “specified subject”. However, the “specified subject” is not limited to the participant J participating in the lesson. For example, the participant J in predetermined outfit in the gym P1, the participant J wearing predetermined clothes, the participant J staying within a range where a distance from the display 11 is less than a predetermined distance, the participant J entering a predetermined room in the gym P1, or a person among the participants J participating in the lesson, the person satisfying a predetermined condition (for example, aged persons and females) may be detected as the “specified subject”.

In the above embodiments, the image display device according to one or more embodiments of the present invention is used for exercise instruction. However, use of the image display device according to one or more embodiments of the present invention is not particularly limited. In a situation where there is a need for confirming biological information and an image (real-time image) of a subject in a remote place at the same time, in particular, in a situation where biological information is easily changed and there is a need for monitoring the biological information, the image display method according to one or more embodiments of the present invention can be effectively utilized.

In the above embodiments, the image display device according to one or more embodiments of the present invention is formed by one computer. That is, in the above embodiments, the case where the functions of the image display device according to one or more embodiments of the present invention are realized by one computer is described with examples. However, the image display device according to one or more embodiments of the present invention may be formed by plural computers. That is, part of the above functions may be realized by another computer. For example, a server computer capable of communicating with the instructor side unit 3 may form the storing section 59.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

### REFERENCE SIGNS LIST

1: Exercise instruction system
2: Participant side unit
3: Instructor side unit
4: First data processing terminal
5: Second data processing terminal (image display device)
11: Display
12: Speaker (device)
13: Camera
14: Microphone
15: Infrared sensor
20: Wearable sensor
30: Biological information storage server
40: Smart band (transmitter)
41: Heartbeat sensor (sensor)
42: Acceleration sensor (action detector)
51: Biological information acquiring section
52: Information analyzing section
53: Image sending section
54: Image displaying section
55: Detecting section
56: Identifying section
57: Position specifying section
58: Change detecting section
59: Storing section
60: Roll call information sending section
61: List making section
100: Exercise instruction system
D1: Transmitted information
D2: Roll call information
I: Instructor (image confirming person)
J: Participant (subject)
L1: List of participant
P1: Gym
P2: Dedicated booth
Tx, Ty: Text box

1. An image display device comprising:
a controller that:
acquires biological information of a subject measured by a sensor at a preset time interval;
displays an image shot by a shooting device on a display;
specifies a position of a figure image included in the shot image, wherein the specified position is within the shot image;
detects a specified subject who is the subject in a predetermined state;
acquires, while detecting the specified subject, the biological information of the specified subject at a second time interval shorter than a first time interval which is the time interval in normal times, and
displays, at the time of displaying the shot image including the figure image of the specified subject on the display, information corresponding to the biological information of the specified subject acquired at the second time interval in a region corresponding to the specified position of the figure image of the specified subject while making the information overlap the figure image.

2. The image display device according to claim 1, wherein the controller updates contents of information displayed as the information corresponding to the biological information of the specified subject while making the information overlap the figure image every time the controller acquires the biological information of the specified subject.

3. The image display device according to claim 1, further comprising:
a storage that stores, by each subject, identification information of the sensor worn by the subject, wherein
the sensor measures the biological information whose magnitude changes according to an activity degree of the subject wearing the sensor, and communicates with the image display device, and
the controller further:
identifies the specified subject who is detected; and
reads the identification information of the sensor associated with the specified subject who is identified out of the storage, and by communicating with the sensor specified by the read identification information, acquires the biological information of the specified subject.

4. The image display device according to claim 1, wherein
the controller detects the specified subject in a predetermined place, and displays the shot image on the display installed in a place separated from the predetermined place.

5. The image display device according to claim 1, wherein:
the controller detects a change in at least one of a face position, a facial direction, and a line of sight of an image confirming person who is in front of the display and confirms the figure image of the specified subject on the display, and
when the controller detects the change, a range of the shot image displayed on the display is shifted according to the change.

6. The image display device according to claim 1, wherein
at the time of displaying the information corresponding to the biological information of the specified subject while making the information overlap the figure image, the controller determines whether the biological information of the specified subject satisfies preset conditions, and displays the information corresponding to the biological information of the specified subject in a display mode corresponding to a determination result.

7. The image display device according to claim 6, wherein:
the controller adds up the biological information acquired at the first time interval in normal times for each subject, and analyzes the biological information for each subject to obtain an analysis result, and
the controller determines whether the biological information of the specified subject satisfies a condition associated with the specified subject among the conditions set for each subject according to the analysis result.

8. The image display device according to claim 1, wherein
when the controller detects plural specified subjects, the controller:
acquires the biological information of the specified subjects measured by the sensors and other information relating to the specified subjects respectively from transmitters prepared for each specified subject, wherein the sensors are mounted on the transmitters,
specifies the position of the figure image of the specified subject who has performed a predetermined action; and
specifies the transmitter that sent the other information relating the specified subject who has performed the predetermined action among the transmitters for each specified subject, and
displays, at the time of displaying the shot image including the figure image of the specified subject who has performed the predetermined action on the display, information corresponding to the biological information acquired from the transmitter specified by the controller in a region corresponding to the position specified by the controller position while making the information overlap the figure image.

9. The image display device according to claim 8, wherein
the controller acquires, respectively from the transmitters prepared for each specified subject, action information generated at the time of detecting actions of the specified subjects by action detectors mounted on the transmitters as the other information, and
the controller specifies the transmitter that sent the action information generated at the time of detecting the predetermined action by the action detector among the transmitters for each specified subject.

10. The image display device according to claim 8, wherein
the controller sends control information for controlling a device installed in a place where there are the plural specified subjects
to make one of the plural specified subjects is encouraged to perform the predetermined action.

11. The image display device according to claim 10, wherein
the controller acquires names a name of each of the specified subjects as the other information from the transmitters prepared for each specified subject,
the controller control information sending section sends the control information for making the installed device generate a sound indicating the name of the one of the plural specified subjects as the control information for controlling the installed device to prompt the one of the plural specified subjects to perform the predetermined action, and
the controller specifies the position of the figure image of the specified subject who has performed a response action to the sound.

12. The image display device according to claim 8, wherein
the controller specifies the position of the figure image of the specified subject who is performing the predetermined action based on data indicating distances between body parts of the specified subject whose figure image is presented in the shot image and a reference position set in a place where the specified subject stays.

13. An image display method comprising:
acquiring, by a controller, biological information of a subject measured by a sensor at a preset time interval;
displaying, by the controller, an image shot by a shooting device on a display;
specifying, by the controller, a position of the figure image included in the shot image, wherein the specified position is within the shot image;
detecting, by the controller, a specified subject who is the subject in a predetermined state;
while detecting the specified subject, acquiring, by a controller, the biological information of the specified subject at a second time interval shorter than a first time interval which is the time interval in normal times;_{;} and
at the time of displaying the shot image including the figure image of the specified subject on the display, displaying, by the controller, information corresponding to the biological information of the specified subject acquired at the second time interval in a region corresponding to the specified position of the figure image of the specified subject while making the information overlap the figure image.
